(19) United States
(12) Patent Application Publication
(10) Pub. No.: US 2026/0017284 A1
(43) Pub. Date: Jan. 15, 2026

(54) DETERMINING LABELS OF INHERITANCE DATASETS USING SIMULATED DATA INSTANCES

(71) Applicant: Ancestry.com DNA, LLC, Lehi, UT (US)

(72) Inventor: Ross Eugene Curtis, Cedar Hills, UT (US)

(21) Appl. No.: 18/767,926

(22) Filed: Jul. 9, 2024

(51) Int. Cl. G06F 16/28

(52) U.S. Cl. CPC G06F 16/285; G06F 16/322; G06N 20/00; G16B 20/00; G16B 30/00

(57) ABSTRACT

Disclosed is a method for determining inheritance labels of users based on inheritance datasets of the users. The method includes generating a plurality of reference panels for a plurality of data-inheritance origins, each reference panel corresponding to a data-inheritance origin and comprising reference-panel datasets representative of the data-inheritance origin. The method constructs a plurality of simulated data trees that are built using the reference-panel datasets that are selected from the plurality of reference panels. The method generates a plurality of simulated inheritance datasets representing a plurality of simulated named entities, each representing a descendant named entity in one of the simulated data trees. The method trains a machine learning model to determine inheritance labels of an inheritance dataset.

### FIELD

The disclosed embodiments relate to assigning labels to data instances, particularly, to determining data inheritances of labels.

### BACKGROUND

Data-inheritance origins may be referred to as origins and describe how data may be inherited from real-world events. Data may be inherited and may evolve based on real-world events that are not always recorded or documented. Yet, while the real-world events may not be completely documented, the change and inheritance of those events may be traceable by comparing data strings among data instances. For example, two data instances may be generated independently and individually reflect the status of their respective named entities or events. The data patterns in the data instances may reflect the natures, histories, or characteristics of data-inheritance sources, such as related or unrelated named entities or events. However, multiple data instances or corresponding named entities or events may be inherited from one or more common sources so that the data instances share some identifiable similarities in the data pattern. As such, the nature of inheritance may be revealed by analyzing and comparing the multiple data instances, and sometimes a large number of data instances. Those real-life events that result in shared data strings among data instances may be referred to as data-inheritance events, even though those real-life events, at the time of the occurrence, may not involve data or data generation at all. For example, the real-life events may be historical events that occurred before the invention of computer or data, but present data instances may still reflect those historical events.

### SUMMARY

Disclosed is a method for determining inheritance labels of users based on inheritance datasets of the users. The method includes generating a plurality of reference panels for a plurality of data-inheritance origins, each reference panel corresponding to a data-inheritance origin and comprising reference-panel datasets representative of the data-inheritance origin. The method constructs a plurality of simulated data trees that are built using the reference-panel datasets that are selected from the plurality of reference panels. The method generates a plurality of simulated inheritance datasets representing a plurality of simulated named entities, each representing a descendant named entity in one of the simulated data trees. The method trains a machine learning model to determine inheritance labels of an inheritance dataset by initiating origin-specific weight parameters in the machine learning model, applying the plurality of simulated inheritance datasets as training samples, applying the machine learning model to predict inheritance labels of the training samples, comparing predicted inheritance labels to actual labels obtained from the plurality of simulated data trees and adjusting the origin-specific weight parameters based on label comparisons.

In another embodiment, a non-transitory computer-readable medium that is configured to store instructions is described. The instructions, when executed by one or more processors, cause the one or more processors to perform a process that includes steps described in the above computer-implemented methods or described in any embodiments of this disclosure. In yet another embodiment, a system may include one or more processors and a storage medium that is configured to store instructions. The instructions, when executed by one or more processors, cause the one or more processors to perform a process that includes steps described in the above computer-implemented methods or described in any embodiments of this disclosure.

### BRIEF DESCRIPTION OF THE DRAWINGS

FIG. 1 illustrates a diagram of a system environment of an example computing system, in accordance with some embodiments.

FIG. 2 is a block diagram of an architecture of an example computing system, in accordance with some embodiments.

FIG. 3 is a flowchart depicting an example process for determining inheritance labels of users based on inheritance datasets, in accordance with some embodiments.

FIG. 4A is a flowchart depicting an example process for selecting a reference-panel individual, in accordance with some embodiments.

FIG. 4B is a graph showing a genetic match correlation, in accordance with some embodiments.

FIG. 4C is a graph showing a match filter, in accordance with some embodiments.

FIG. 5A is a flowchart depicting an example process for generating training samples from a plurality of simulated individuals, in accordance with some embodiments.

FIG. 5B shows the proportions of a simulated tree, in accordance with some embodiments.

FIG. 5C shows a simulated data tree and genetic distribution data of ancestral categories of the simulated tree, in accordance with some embodiments.

FIG. 6 is an example of an inter-window HMM, in accordance with some embodiments.

FIG. 7A is a flowchart illustrating a process for calculating emission probability, in accordance with some embodiments.

FIG. 7B shows the results of the machine learning model before the step of initiating origin-specific weight parameters, in accordance with some embodiments.

FIG. 7C shows the results of the machine learning model after the step of initiating origin-specific weight parameters, in accordance with some embodiments.

FIG. 7D shows examples of weight parameters associated with specific populations, in accordance with some embodiments.

FIG. 8 is a block diagram of an example computing device, in accordance with some embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTIONS

The figures (FIGs.) and the following description relate to preferred embodiments by way of illustration only. One of skill in the art may recognize alternative embodiments of the structures and methods disclosed herein as viable alternatives that may be employed without departing from the principles of what is disclosed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

Disclosed are techniques for determining inheritance labels of users based on inheritance datasets of the users. The method generates a plurality of reference panels for a plurality of data-inheritance origins. The method constructs a plurality of simulated data trees that are built using reference-panel individuals. The method generates a plurality of simulated inheritance datasets representing a plurality of simulated individuals each representing a descendant in one of the simulated data trees. The method trains a machine learning model to determine inheritance labels of users based on inheritance datasets of the users by initiating origin-specific weight parameters in the machine learning model, applying the plurality of simulated inheritance datasets as training samples, applying the machine learning model to predict inheritance labels of the training samples, comparing predicted inheritance labels to actual labels obtained from the plurality of simulated data trees and adjusting the origin-specific weight parameters based on label comparisons.

### Example System Environment

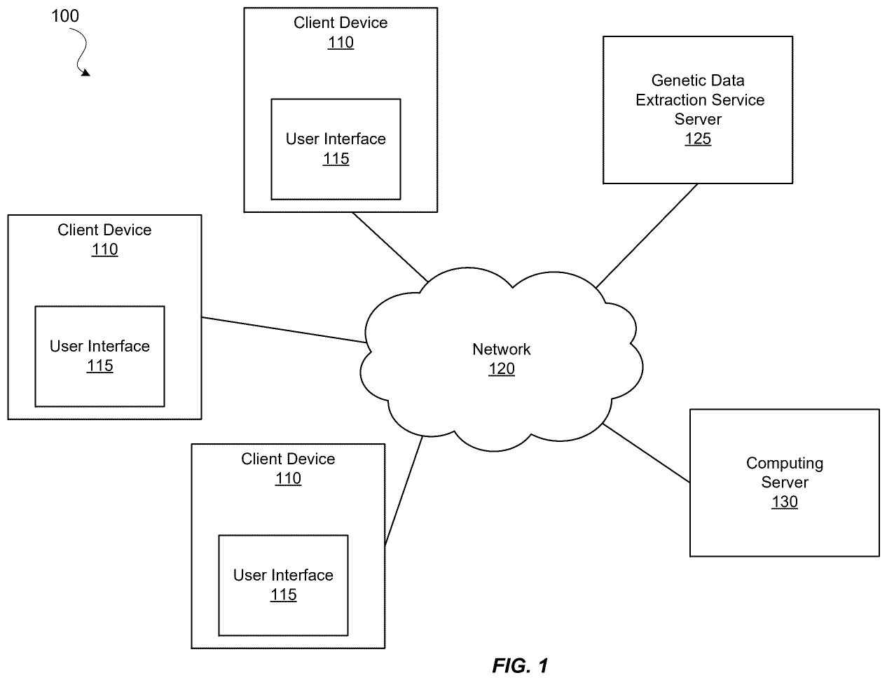

FIG. 1 illustrates a diagram of a system environment 100 of an example computing server 130, in accordance with some embodiments. The system environment 100 shown in FIG. 1 includes one or more client devices 110, a network 120, a genetic data extraction service server 125, and a computing server 130. In various embodiments, the system environment 100 may include fewer or additional components. The system environment 100 may also include different components.

The client devices 110 are one or more computing devices capable of receiving user input as well as transmitting and/or receiving data via a network 120. Example computing devices include desktop computers, laptop computers, personal digital assistants (PDAs), smartphones, tablets, wearable electronic devices (e.g., smartwatches), smart household appliances (e.g., smart televisions, smart speakers, smart home hubs), Internet of Things (IoT) devices or other suitable electronic devices. A client device 110 communicates to other components via the network 120. Users may be customers of the computing server 130 or any individuals who access the system of the computing server 130, such as an online website or a mobile application. In some embodiments, a client device 110 executes an application that launches a graphical user interface (GUI) for a user of the client device 110 to interact with the computing server 130. The GUI may be an example of a user interface 115. A client device 110 may also execute a web browser application to enable interactions between the client device 110 and the computing server 130 via the network 120. In another embodiment, the user interface 115 may take the form of a software application published by the computing server 130 and installed on the user device 110. In yet another embodiment, a client device 110 interacts with the computing server 130 through an application programming interface (API) running on a native operating system of the client device 110, such as IOS or ANDROID.

The network 120 provides connections to the components of the system environment 100 through one or more sub-networks, which may include any combination of local area and/or wide area networks, using both wired and/or wireless communication systems. In some embodiments, a network 120 uses standard communications technologies and/or protocols. For example, a network 120 may include communication links using technologies such as Ethernet, 802.11, worldwide interoperability for microwave access (WiMAX), 3G, 4G, Long Term Evolution (LTE), 5G, code division multiple access (CDMA), digital subscriber line (DSL), etc. Examples of network protocols used for communicating via the network 120 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over a network 120 may be represented using any suitable format, such as hypertext markup language (HTML) or extensible markup language (XML). In some embodiments, all or some of the communication links of a network 120 may be encrypted using any suitable technique or techniques such as secure sockets layer (SSL), transport layer security (TLS), virtual private networks (VPNs), Internet Protocol security (IPsec), etc. The network 120 also includes links and packet-switching networks such as the Internet.

Individuals, who may be customers of a company operating the computing server 130, provide biological samples for analysis of their genetic data. Individuals may also be referred to as users. In some embodiments, an individual uses a sample collection kit to provide a biological sample (e.g., saliva, blood, hair, tissue) from which genetic data is extracted and determined according to nucleotide processing techniques such as microarray, amplification and/or sequencing. Microarray may include immobilizing probe DNA sequences, onto a solid surface such as a glass slide. Target DNA samples, labeled with fluorescent tags, are then applied to the microarray surface. Through complementary base pairing, the labeled DNA binds to its corresponding probe on the microarray. By detecting the fluorescence emitted by the labeled DNA, genetic data may be extracted. Amplification may include using polymerase chain reaction (PCR) to amplify segments of nucleotide samples. Sequencing may include sequencing of deoxyribonucleic acid (DNA) sequencing, ribonucleic acid (RNA) sequencing, etc. Suitable sequencing techniques may include Sanger sequencing and massively parallel sequencing such as various next-generation sequencing (NGS) techniques including whole genome sequencing, pyrosequencing, sequencing by synthesis, sequencing by ligation, and ion semiconductor sequencing. In some embodiments, a set of SNPs (e.g., 300,000) that are shared between different array platforms (e.g., Illumina OmniExpress Platform and Illumina HumanHap 650Y Platform) may be obtained as genetic data. Genetic data extraction service server 125 receives biological samples from users of the computing server 130. The genetic data extraction service server 125 extracts genetic data from the samples and the data may take the form of a set of SNPs. The genetic data extraction service server 125 generates the genetic data of the individuals based on sequencing or microarray results. The genetic data may include data generated from DNA or RNA and may include base pairs from coding and/or noncoding regions of DNA.

The genetic data may take different forms and include information regarding various biomarkers of an individual. For example, in some embodiments, the genetic data may be the base pair sequence of an individual. The base pair sequence may include the whole genome or a part of the genome such as certain genetic loci of interest. In another embodiment, the genetic data extraction service server 125 may determine genotypes from DNA identification results, for example by identifying genotype values of single nucleotide polymorphisms (SNPs) present within the DNA. The results in this example may include a sequence of genotypes corresponding to various SNP sites. A SNP site may also be referred to as a SNP loci. A genetic locus is a segment of a genetic sequence. A locus can be a single site or a longer stretch. The segment can be a single base long or multiple bases long. In some embodiments, the genetic data extraction service server 125 may perform data pre-processing of the genetic data to convert raw sequences of base pairs to sequences of genotypes at target SNP sites. Since a typical human genome may differ from a reference human genome at only several million SNP sites (as opposed to billions of base pairs in the whole genome), the genetic data extraction service server 125 may extract only the genotypes at a set of target SNP sites and transmit the extracted data to the computing server 130 as the inheritance dataset of an individual. SNPs, base pair sequences, genotypes, haplotypes, RNA sequences, protein sequences, and phenotypes are examples of biomarkers. In some embodiments, each SNP site may have two readings that are heterozygous.

The computing server 130 performs various analyses of the genetic data, genealogy data, and users' survey responses to generate results regarding the phenotypes and genealogy of users of computing server 130. Depending on the embodiments, the computing server 130 may also be referred to as an online server, a personal genetic service server, a genealogy server, a family tree building server, and/or a social networking system. The computing server 130 receives genetic data from the genetic data extraction service server 125 and stores the genetic data in the data store of the computing server 130. The computing server 130 may analyze the data to generate results regarding the genetics or genealogy of users. The results regarding the genetics or genealogy of users may include the ethnicity compositions of users, paternal and maternal genetic analysis, identification or suggestion of potential family relatives, ancestor information, analyses of DNA data, potential or identified traits such as phenotypes of users (e.g., diseases, appearance traits, other genetic characteristics, and other non-genetic characteristics including social characteristics), etc. The computing server 130 may present or cause the user interface 115 to present the results to the users through a GUI displayed on the client device 110. The results may include graphical elements, textual information, data, charts, and other elements such as family trees.

In some embodiments, the computing server 130 also allows various users to create one or more genealogical profiles of the user. The genealogical profile may include a list of individuals (e.g., ancestors, relatives, friends, and other people of interest) who are added or selected by the user or suggested by the computing server 130 based on the genealogical records and/or genetic records. The user interface 115 controlled by or in communication with the computing server 130 may display the individuals in a list or as a family tree such as in the form of a pedigree chart. In some embodiments, subject to the user's privacy setting and authorization, the computing server 130 may allow information generated from the user's inheritance dataset to be linked to the user profile and to one or more of the family trees. The users may also authorize the computing server 130 to analyze their inheritance dataset and allow their profiles to be discovered by other users.

### Example Computing Server Architecture

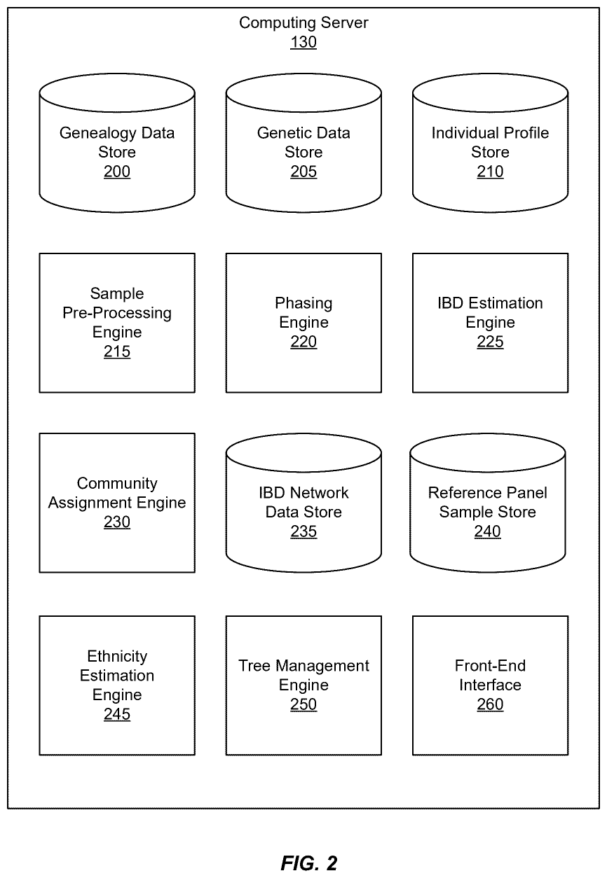

FIG. 2 is a block diagram of the architecture of an example computing server 130, in accordance with some embodiments. In the embodiment shown in FIG. 2, the computing server 130 includes a genealogy data store 200, a genetic data store 205, an individual profile store 210, a sample pre-processing engine 215, a phasing engine 220, an identity by descent (IBD) estimation engine 225, a community assignment engine 230, an IBD network data store 235, a reference panel sample store 240, an ethnicity estimation engine 245, a front-end interface 260, and a tree management engine 250. The functions of the computing server 130 may be distributed among the elements in a different manner than described. In various embodiments, the computing server 130 may include different components and fewer or additional components. Each of the various data stores may be a single storage device, a server controlling multiple storage devices, or a distributed network that is accessible through multiple nodes (e.g., a cloud storage system).

The computing server 130 stores various data of different individuals, including genetic data, genealogy data, and survey response data. The computing server 130 processes the genetic data of users to identify shared identity-by-descent (IBD) segments between individuals. The genealogy data and survey response data may be part of user profile data. The amount and type of user profile data stored for each user may vary based on the information of a user, which is provided by the user as she creates an account and profile at a system operated by the computing server 130 and continues to build her profile, family tree, and social network at the system and to link her profile with her genetic data. Users may provide data via the user interface 115 of a client device 110. Initially and as a user continues to build her genealogical profile, the user may be prompted to answer questions related to the basic information of the user (e.g., name, date of birth, birthplace, etc.) and later on more advanced questions that may be useful for obtaining additional genealogy data. The computing server 130 may also include survey questions regarding various traits of the users such as the users' phenotypes, characteristics, preferences, habits, lifestyle, environment, etc.

Genealogy data may be stored in the genealogy data store 200 and may include various types of data that are related to tracing family relatives of users. Examples of genealogy data include names (first, last, middle, suffixes), gender, birth locations, date of birth, date of death, marriage information, spouse's information kinships, family history, dates and places for life events (e.g., birth and death), other vital data, and the like. In some instances, family history can take the form of a pedigree of an individual (e.g., the recorded relationships in the family). The family tree information associated with an individual may include one or more specified nodes. Each node in the family tree represents the individual, an ancestor of the individual who might have passed down genetic material to the individual, and the individual's other relatives including siblings, cousins, and offspring in some cases. Genealogy data may also include connections and relationships among users of the computing server 130. The information related to the connections between a user and her relatives that may be associated with a family tree may also be referred to as pedigree data or family tree data.

In addition to user-input data, genealogy data may also take other forms that are obtained from various sources such as public records and third-party data collectors. For example, genealogical records from public sources include birth records, marriage records, death records, census records, court records, probate records, adoption records, obituary records, etc. Likewise, genealogy data may include data from one or more family trees of an individual, the Ancestry World Tree system, a Social Security Death Index database, the World Family Tree system, a birth certificate database, a death certificate database, a marriage certificate database, an adoption database, a draft registration database, a veterans database, a military database, a property records database, a census database, a voter registration database, a phone database, an address database, a newspaper database, an immigration database, a family history records database, a local history records database, a business registration database, a motor vehicle database, and the like.

Furthermore, the genealogy data store 200 may also include relationship information inferred from the genetic data stored in the genetic data store 205 and information received from the individuals. For example, the relationship information may indicate which individuals are genetically related, how they are related, how many generations back they share common ancestors, lengths and locations of IBD segments shared, which genetic communities an individual is a part of, variants carried by the individual, and the like.

The computing server 130 maintains inheritance datasets of individuals in the genetic data store 205. An inheritance dataset of an individual may be a digital dataset of nucleotide data (e.g., SNP data) and corresponding metadata. For example, an inheritance dataset may be genetic data extracted by the genetic data extraction service server 125. An inheritance dataset may contain data on the whole or portions of an individual's genome. The genetic data store 205 may store a pointer to a location associated with the genealogy data store 200 associated with the individual. An inheritance dataset may take different forms. In some embodiments, an inheritance dataset may take the form of a base pair sequence of the sequencing result of an individual. A base pair sequence dataset may include the whole genome of the individual (e.g., obtained from a whole-genome sequencing) or some parts of the genome (e.g., genetic loci of interest). A microarray data may take the form of SNP data at target positions in the genome.

In another embodiment, an inheritance dataset may take the form of sequences of genetic markers. Examples of genetic markers may include target SNP sites (e.g., allele sites) filtered from the DNA identification results. A SNP site that is a single base pair long may also be referred to as a SNP locus. A SNP site may be associated with a unique identifier. The inheritance dataset may be in the form of diploid data that includes a sequence of genotypes, such as genotypes at the target SNP site, or the whole base pair sequence that includes genotypes at known SNP sites and other base pair sites that are not commonly associated with known SNPs. The diploid dataset may be referred to as a genotype dataset or a genotype sequence. Genotype may have a different meaning in various contexts. In one context, an individual's genotype may refer to a collection of diploid alleles of an individual. In other contexts, a genotype may be a pair of alleles present on two chromosomes for an individual at a given genetic marker such as a SNP site.

Genotype data for a SNP site may include a pair of alleles. The pair of alleles may be homozygous (e.g., A-A or G-G) or heterozygous (e.g., A-T, C-T). Instead of storing the actual nucleotides, the genetic data store 205 may store genetic data that are converted to bits. For a given SNP site, oftentimes only two nucleotide alleles (instead of all 4) are observed. As such, a 2-bit number may represent a SNP site. For example, 00 may represent homozygous first alleles, 11 may represent homozygous second alleles, and 01 or 10 may represent heterozygous alleles. A separate library may store what nucleotide corresponds to the first allele and what nucleotide corresponds to the second allele at a given SNP site.

A diploid dataset may also be phased into two sets of haploid data, one corresponding to a first parent side and another corresponding to a second parent side. The phased datasets may be referred to as haplotype datasets or haplotype sequences. Similar to genotype, haplotype may have a different meaning in various contexts. In one context, a haplotype may also refer to a collection of alleles that corresponds to a genetic segment. In other contexts, a haplotype may refer to a specific allele at a SNP site. For example, a sequence of haplotypes may refer to a sequence of alleles of an individual that are inherited from a parent.

The individual profile store 210 stores profiles and related metadata associated with various individuals appeared in the computing server 130. A computing server 130 may use unique individual identifiers to identify various users and other non-users that might appear in other data sources such as ancestors or historical persons who appear in any family tree or genealogy database. A unique individual identifier may be a hash of certain identification information of an individual, such as a user's account name, user's name, date of birth, location of birth, or any suitable combination of the information. The profile data related to an individual may be stored as metadata associated with an individual's profile. For example, the unique individual identifier and the metadata may be stored as a key-value pair using the unique individual identifier as a key.

An individual's profile data may include various kinds of information related to the individual. The metadata about the individual may include one or more pointers associating inheritance datasets such as genotype and phased haplotype data of the individual that are saved in the genetic data store 205. The metadata about the individual may also be individual information related to family trees and pedigree datasets that include the individual. The profile data may further include declarative information about the user that was authorized by the user to be shared and may also include information inferred by the computing server 130. Other examples of information stored in a user profile may include biographic, demographic, and other types of descriptive information such as work experience, educational history, gender, hobbies, preferences, location and the like. In some embodiments, the user profile data may also include one or more photos of the users and photos of relatives (e.g., ancestors) of the users that are uploaded by the users. A user may authorize the computing server 130 to analyze one or more photos to extract information, such as the user's or relative's appearance traits (e.g., blue eyes, curved hair, etc.), from the photos. The appearance traits and other information extracted from the photos may also be saved in the profile store. In some cases, the computing server may allow users to upload many different photos of the users, their relatives, and even friends. User profile data may also be obtained from other suitable sources, including historical records (e.g., records related to an ancestor), medical records, military records, photographs, other records indicating one or more traits, and other suitable recorded data.

For example, the computing server 130 may present various survey questions to its users from time to time. The responses to the survey questions may be stored at individual profile store 210. The survey questions may be related to various aspects of the users and the users' families. Some survey questions may be related to users' phenotypes, while other questions may be related to the environmental factors of the users.

Survey questions may concern health or disease-related phenotypes, such as questions related to the presence or absence of genetic diseases or disorders, inheritable diseases or disorders, or other common diseases or disorders that have a family history as one of the risk factors, questions regarding any diagnosis of increased risk of any diseases or disorders, and questions concerning wellness-related issues such as a family history of obesity, family history of causes of death, etc. The diseases identified by the survey questions may be related to single-gene diseases or disorders that are caused by a single-nucleotide variant, an insertion, or a deletion. The diseases identified by the survey questions may also be multifactorial inheritance disorders that may be caused by a combination of environmental factors and genes. Examples of multifactorial inheritance disorders may include heart disease, Alzheimer's disease, diabetes, cancer, and obesity. The computing server 130 may obtain data on a user's disease-related phenotypes from survey questions about the health history of the user and her family and also from health records uploaded by the user.

Survey questions also may be related to other types of phenotypes such as appearance traits of the users. A survey regarding appearance traits and characteristics may include questions related to eye color, iris pattern, freckles, chin types, finger length, dimple chin, earlobe types, hair color, hair curl, skin pigmentation, susceptibility to skin burn, bitter taste, male baldness, baldness pattern, presence of unibrow, presence of wisdom teeth, height, and weight. A survey regarding other traits also may include questions related to users' taste and smell such as the ability to taste bitterness, asparagus smell, cilantro aversion, etc. A survey regarding traits may further include questions related to users' body conditions such as lactose tolerance, caffeine consumption, malaria resistance, norovirus resistance, muscle performance, alcohol flush, etc. Other survey questions regarding a person's physiological or psychological traits may include vitamin traits and sensory traits such as the ability to sense an asparagus metabolite. Traits may also be collected from historical records, electronic health records and electronic medical records.

The computing server 130 also may present various survey questions related to the environmental factors of users. In this context, an environmental factor may be a factor that is not directly connected to the genetics of the users. Environmental factors may include users' preferences, habits, and lifestyles. For example, a survey regarding users' preferences may include questions related to things and activities that users like or dislike, such as types of music a user enjoys, dancing preference, party-going preference, certain sports that a user plays, video game preferences, etc. Other questions may be related to the users' diet preferences such as like or dislike a certain type of food (e.g., ice cream, egg). A survey related to habits and lifestyle may include questions regarding smoking habits, alcohol consumption and frequency, daily exercise duration, sleeping habits (e.g., morning person versus night person), sleeping cycles and problems, hobbies, and travel preferences. Additional environmental factors may include diet amount (calories, macronutrients), physical fitness abilities (e.g., stretching, flexibility, heart rate recovery), family type (adopted family or not, has siblings or not, lived with extended family during childhood), property and item ownership (has home or rents, has a smartphone or doesn't, has a car or doesn't).

Surveys also may be related to other environmental factors such as geographical, social-economic, or cultural factors. Geographical questions may include questions related to the birth location, family migration history, town, or city of users' current or past residence. Social-economic questions may be related to users' education level, income, occupations, self-identified demographic groups, etc. Questions related to culture may concern users' native language, language spoken at home, customs, dietary practices, etc. Other questions related to users' cultural and behavioral questions are also possible.

For any survey questions asked, the computing server 130 may also ask an individual the same or similar questions regarding the traits and environmental factors of the ancestors, family members, other relatives or friends of the individual. For example, a user may be asked about the native language of the user and the native languages of the user's parents and grandparents. A user may also be asked about the health history of his or her family members.

In addition to storing the survey data in the individual profile store 210, the computing server 130 may store some responses that correspond to data related to genealogical and genetics respectively to genealogy data store 200 and genetic data store 205.

The user profile data, photos of users, survey response data, the genetic data, and the genealogy data may be subject to the privacy and authorization setting of the users to specify any data related to the users that can be accessed, stored, obtained, or otherwise used. For example, when presented with a survey question, a user may select to answer or skip the question. The computing server 130 may present users from time to time information regarding users' selection of the extent of information and data shared. The computing server 130 also may maintain and enforce one or more privacy settings for users in connection with the access of the user profile data, photos, genetic data, and other sensitive data. For example, the user may pre-authorize the access to the data and may change the setting as wished. The privacy settings also may allow a user to specify (e.g., by opting out, by not opting in) whether the computing server 130 may receive, collect, log, or store particular data associated with the user for any purpose. A user may restrict her data at various levels. For example, on one level, the data may not be accessed by the computing server 130 for purposes other than displaying the data in the user's own profile. On another level, the user may authorize anonymization of her data and participate in studies and research conducted by the computing server 130 such as a large-scale genetic study. On yet another level, the user may turn some portions of her genealogy data public to allow the user to be discovered by other users (e.g., potential relatives) and be connected to one or more family trees. Access or sharing of any information or data in the computing server 130 may also be subject to one or more similar privacy policies. A user's data and content objects in the computing server 130 may also be associated with different levels of restriction. The computing server 130 may also provide various notification features to inform and remind users of their privacy and access settings. For example, when privacy settings for a data entry allow a particular user or other entities to access the data, the data may be described as being “visible,” “public,” or other suitable labels, contrary to a “private” label.

In some cases, the computing server 130 may have heightened privacy protection on certain types of data and data related to certain vulnerable groups. In some cases, the heightened privacy settings may strictly prohibit the use, analysis, and sharing of data related to a certain vulnerable group. In other cases, the heightened privacy settings may specify that data subject to those settings require prior approval for access, publication, or other use. In some cases, the computing server 130 may provide heightened privacy as a default setting for certain types of data, such as genetic data or any data that the user marks as sensitive. The user may opt in to sharing those data or change the default privacy settings. In other cases, the heightened privacy settings may apply across the board for all data of certain groups of users. For example, if computing server 130 determines that the user is a minor or has recognized that a picture of a minor is uploaded, the computing server 130 may designate all profile data associated with the minor as sensitive. In those cases, the computing server 130 may have one or more extra steps in seeking and confirming any sharing or use of the sensitive data.

In some embodiments, the individual profile store 210 may be a large-scale data store. In some embodiments, the individual profile store 210 may include at least 10,000 data records in the form of user profiles and each user profile may be associated with one or more inheritance datasets and one or more genealogical data entries. In some embodiments, the individual profile store 210 may include at least 50,000 data records in the form of user profiles and each user profile may be associated with one or more inheritance datasets and one or more genealogical data entries. In some embodiments, the individual profile store 210 may include at least 100,000 data records in the form of user profiles and each user profile may be associated with one or more inheritance datasets and one or more genealogical data entries. In some embodiments, the individual profile store 210 may include at least 500,000 data records in the form of user profiles and each user profile may be associated with one or more inheritance datasets and one or more genealogical data entries. In some embodiments, the individual profile store 210 may include at least 1,000,000 data records in the form of user profiles and each user profile may be associated with one or more inheritance datasets and one or more genealogical data entries. In some embodiments, the individual profile store 210 may include at least 2,000,000 data records in the form of user profiles and each user profile may be associated with one or more inheritance datasets and one or more genealogical data entries. In some embodiments, the individual profile store 210 may include at least 5,000,000 data records in the form of user profiles and each user profile may be associated with one or more inheritance datasets and one or more genealogical data entries. In some embodiments, the individual profile store 210 may include at least 10,000,000 data records in the form of user profiles and each user profile may be associated with one or more inheritance datasets and one or more genealogical data entries.

The sample pre-processing engine 215 receives and pre-processes data received from various sources to change the data into a format used by the computing server 130. For genealogy data, the sample pre-processing engine 215 may receive data from an individual via the user interface 115 of the client device 110. To collect the user data (e.g., genealogical and survey data), the computing server 130 may cause an interactive user interface on the client device 110 to display interface elements in which users can provide genealogy data and survey data. Additional data may be obtained from scans of public records. The data may be manually provided or automatically extracted via, for example, optical character recognition (OCR) performed on census records, town or government records, or any other item of printed or online material. Some records may be obtained by digitalizing written records such as older census records, birth certificates, death certificates, etc.

The sample pre-processing engine 215 may also receive raw data from the genetic data extraction service server 125. The genetic data extraction service server 125 may perform laboratory analysis of biological samples of users and generate sequencing results in the form of digital data. The sample pre-processing engine 215 may receive the raw inheritance datasets from the genetic data extraction service server 125. Most of the mutations that are passed down to descendants are related to single-nucleotide polymorphism (SNP). SNP is a substitution of a single nucleotide that occurs at a specific position in the genome. The sample pre-processing engine 215 may convert the raw base pair sequence into a sequence of genotypes of target SNP sites. Alternatively, the pre-processing of this conversion may be performed by the genetic data extraction service server 125. The sample pre-processing engine 215 identifies autosomal SNPs in an individual's inheritance dataset. In some embodiments, the SNPs may be autosomal SNPs. In some embodiments, 700,000 SNPs may be identified in an individual's data and may be stored in genetic data store 205. Alternatively, in some embodiments, an inheritance dataset may include at least 10,000 SNP sites. In another embodiment, an inheritance dataset may include at least 100,000 SNP sites. In yet another embodiment, an inheritance dataset may include at least 300,000 SNP sites. In yet another embodiment, an inheritance dataset may include at least 1,000,000 SNP sites. The sample pre-processing engine 215 may also convert the nucleotides into bits. The identified SNPs, in bits or in other suitable formats, may be provided to the phasing engine 220 which phases the individual's diploid genotypes to generate a pair of haplotypes for each user.

The phasing engine 220 phases a diploid inheritance dataset into a pair of haploid inheritance datasets and may perform imputation of SNP values at certain sites whose alleles are missing. An individual's haplotype may refer to a collection of alleles (e.g., a sequence of alleles) that are inherited from a parent.

Phasing may include a process of determining the assignment of alleles (particularly heterozygous alleles) to chromosomes. Owing to conditions and other constraints in sequencing or microarray, a DNA identification result often includes data regarding a pair of alleles at a given SNP locus of a pair of chromosomes but may not be able to distinguish which allele belongs to which specific chromosome. The phasing engine 220 uses a genotype phasing algorithm to assign one allele to a first chromosome and another allele to another chromosome. The genotype phasing algorithm may be developed based on an assumption of linkage disequilibrium (LD), which states that haplotype in the form of a sequence of alleles tends to cluster together. The phasing engine 220 is configured to generate phased sequences that are also commonly observed in many other samples. Put differently, haplotype sequences of different individuals tend to cluster together. A haplotype-cluster model may be generated to determine the probability distribution of a haplotype that includes a sequence of alleles. The haplotype-cluster model may be trained based on labeled data that includes known phased haplotypes from a trio (parents and a child). A trio is used as a training sample because the correct phasing of the child is almost certain by comparing the child's genotypes to the parent's inheritance datasets. The haplotype-cluster model may be generated iteratively along with the phasing process with a large number of unphased genotype datasets. The haplotype-cluster model may also be used to impute one or more missing data.

By way of example, the phasing engine 220 may use a directed acyclic graph model such as a hidden Markov model (HMM) to perform the phasing of a target genotype dataset. The directed acyclic graph may include multiple levels, each level having multiple nodes representing different possibilities of haplotype clusters. An emission probability of a node, which may represent the probability of having a particular haplotype cluster given an observation of the genotypes may be determined based on the probability distribution of the haplotype-cluster model. A transition probability from one node to another may be initially assigned to a non-zero value and be adjusted as the directed acyclic graph model and the haplotype-cluster model are trained. Various paths are possible in traversing different levels of the directed acyclic graph model. The phasing engine 220 determines a statistically likely path, such as the most probable path or a probable path that is at least more likely than 95% of other possible paths, based on the transition probabilities and the emission probabilities. A suitable dynamic programming algorithm such as the Viterbi algorithm may be used to determine the path. The determined path may represent the phasing result. U.S. Pat. No. 10,679,729, entitled “Haplotype Phasing Models,” granted on Jun. 9, 2020, describes example embodiments of haplotype phasing.

A phasing algorithm may also generate phasing result that has a long genomic distance accuracy and cross-chromosome accuracy in terms of haplotype separation. For example, in some embodiments, an IBD-phasing algorithm may be used, which is described in further detail in U.S. Patent Application Publication No. US 2021/0034647, entitled “Clustering of Matched Segments to Determine Linkage of Dataset in a Database,” published on Feb. 4, 2021. For example, the computing server 130 may receive a target individual genotype dataset and a plurality of additional individual genotype datasets that include haplotypes of additional individuals. For example, the additional individuals may be reference panels or individuals who are linked (e.g., in a family tree) to the target individual. The computing server 130 may generate a plurality of sub-cluster pairs of first parental groups and second parental groups. Each sub-cluster pair may be in a window. The window may correspond to a genomic segment and has a similar concept of window used in the ethnicity estimation engine 245 and the rest of the disclosure related to HMMs, but how windows are precisely divided and defined may be the same or different in the phasing engine 220 and in an HMM. Each sub-cluster pair may correspond to a genetic locus. In some embodiments, each sub-cluster pair may have a first parental group that includes a first set of matched haplotype segments selected from the plurality of additional individual datasets and a second parental group that includes a second set of matched haplotype segments selected from the plurality of additional individual datasets. The computing server 130 may generate a super-cluster of a parental side by linking the first parental groups and the second parental groups across a plurality of genetic loci (across a plurality of sub-cluster pairs). Generating the super-cluster of the parental side may include generating a candidate parental side assignment of parental groups across a set of sub-cluster pairs that represent a set of genetic loci in the plurality of genetic loci. The computing server 130 may determine the number of common additional individual genotype datasets that are classified in the candidate parental side assignment. The computing server 130 may determine the candidate parental side assignment to be part of the super-cluster based on the number of common additional individual genotype datasets. Any suitable algorithms may be used to generate the super-cluster, such as a heuristic scoring approach, a bipartite graph approach, or another suitable approach. The computing server 130 may generate a haplotype phasing of the target individual from the super-cluster of the parental side.

The IBD estimation engine 225 estimates the amount of shared genetic segments between a pair of individuals based on phased genotype data (e.g., haplotype datasets) that are stored in the genetic data store 205. IBD segments may be segments identified in a pair of individuals that are putatively determined to be inherited from a common ancestor. The IBD estimation engine 225 retrieves a pair of haplotype datasets for each individual. The IBD estimation engine 225 may divide each haplotype dataset sequence into a plurality of windows. Each window may include a fixed number of SNP sites (e.g., about 100 SNP sites). The IBD estimation engine 225 identifies one or more seed windows in which the alleles at all SNP sites in at least one of the phased haplotypes between two individuals are identical. The IBD estimation engine 225 may expand the match from the seed windows to nearby windows until the matched windows reach the end of a chromosome or until a homozygous mismatch is found, which indicates the mismatch is not attributable to potential errors in phasing or imputation. The IBD estimation engine 225 determines the total length of matched segments, which may also be referred to as IBD segments. The length may be measured in the genetic distance in the unit of centimorgans (cM). A unit of centimorgan may be a genetic length. For example, two genomic positions that are one cM apart may have a 1% chance during each meiosis of experiencing a recombination event between the two positions. The computing server 130 may save data regarding individual pairs who share a length of IBD segments exceeding a predetermined threshold (e.g., 6 cM), in a suitable data store such as in the genealogy data store 200. U.S. Pat. No. 10,114,922, entitled “Identifying Ancestral Relationships Using a Continuous stream of Input,” granted on Oct. 30, 2018, and U.S. Pat. No. 10,720,229, entitled “Reducing Error in Predicted Genetic Relationships,” granted on Jul. 21, 2020, describe example embodiments of IBD estimation.

Typically, individuals who are closely related share a relatively large number of IBD segments, and the IBD segments tend to have longer lengths (individually or in aggregate across one or more chromosomes). In contrast, individuals who are more distantly related share relatively fewer IBD segments, and these segments tend to be shorter (individually or in aggregate across one or more chromosomes). For example, while close family members often share upwards of 71 cM of IBD (e.g., third cousins), more distantly related individuals may share less than 12 cM of IBD. The extent of relatedness in terms of IBD segments between two individuals may be referred to as IBD affinity. For example, the IBD affinity may be measured in terms of the length of IBD segments shared between two individuals.

Community assignment engine 230 assigns individuals to one or more genetic communities based on the genetic data of the individuals. A genetic community may correspond to an ethnic origin or a group of people descended from a common ancestor. The granularity of genetic community classification may vary depending on embodiments and methods used to assign communities. For example, in some embodiments, the communities may be African, Asian, European, etc. In another embodiment, the European community may be divided into Irish, German, Swedes, etc. In yet another embodiment, the Irish may be further divided into Irish in Ireland, Irish who immigrated to America in 1800, Irish who immigrated to America in 1900, etc. The community classification may also depend on whether a population is admixed or unadmixed. For an admixed population, the classification may further be divided based on different ethnic origins in a geographical region.

Community assignment engine 230 may assign individuals to one or more genetic communities based on their inheritance datasets using machine learning models trained by unsupervised learning or supervised learning. In an unsupervised approach, the community assignment engine 230 may generate data representing a partially connected undirected graph. In this approach, the community assignment engine 230 represents individuals as nodes. Some nodes are connected by edges whose weights are based on IBD affinity between two individuals represented by the nodes. For example, if the total length of two individuals' shared IBD segments does not exceed a predetermined threshold, the nodes are not connected. The edges connecting two nodes are associated with weights that are measured based on the IBD affinities. The undirected graph may be referred to as an IBD network. The community assignment engine 230 uses clustering techniques such as modularity measurement (e.g., the Louvain method) to classify nodes into different clusters in the IBD network. Each cluster may represent a community. The community assignment engine 230 may also determine sub-clusters, which represent sub-communities. The computing server 130 saves the data representing the IBD network and clusters in the IBD network data store 235. U.S. Pat. No. 10,223,498, entitled “Discovering Population Structure from Patterns of Identity-By-Descent,” granted on Mar. 5, 2019, describes example embodiments of community detection and assignment.

The community assignment engine 230 may also assign communities using supervised techniques. For example, inheritance datasets of known genetic communities (e.g., individuals with confirmed ethnic origins) may be used as training sets that have labels of the genetic communities. Supervised machine learning classifiers, such as logistic regressions, support vector machines, random forest classifiers, and neural networks may be trained using the training set with labels. A trained classifier may distinguish binary or multiple classes. For example, a binary classifier may be trained for each community of interest to determine whether a target individual's inheritance dataset belongs or does not belong to the community of interest. A multi-class classifier such as a neural network may also be trained to determine whether the target individual's inheritance dataset most likely belongs to one of several possible genetic communities.

Reference panel sample store 240 stores reference panel samples for different genetic communities. A reference panel sample is the genetic data of an individual whose genetic data is the most representative of a genetic community. The genetic data of individuals with the typical alleles of a genetic community may serve as reference panel samples. For example, some alleles of genes may be over-represented (e.g., being highly common) in a genetic community. Some inheritance datasets include alleles that are commonly present among members of the community. Reference panel samples may be used to train various machine learning models in classifying whether a target inheritance dataset belongs to a community, determining the ethnic composition of an individual, and determining the accuracy of any genetic data analysis, such as by computing a posterior probability of a classification result from a classifier.

A reference panel sample may be identified in different ways. In some embodiments, an unsupervised approach in community detection may apply the clustering algorithm recursively for each identified cluster until the sub-clusters contain a number of nodes that are smaller than a threshold (e.g., containing fewer than 1000 nodes). For example, the community assignment engine 230 may construct a full IBD network that includes a set of individuals represented by nodes and generate communities using clustering techniques. The community assignment engine 230 may randomly sample a subset of nodes to generate a sampled IBD network. The community assignment engine 230 may recursively apply clustering techniques to generate communities in the sampled IBD network. The sampling and clustering may be repeated for different randomly generated IBD networks for various runs. Nodes that are consistently assigned to the same genetic community when sampled in various runs may be classified as a reference panel sample. The community assignment engine 230 may measure the consistency in terms of a predetermined threshold. For example, if a node is classified to the same community 95% (or another suitable threshold) of the times the node is sampled, the inheritance dataset corresponding to the individual represented by the node may be regarded as a reference panel sample. Additionally, or alternatively, the community assignment engine 230 may select N most consistently assigned nodes as a reference panel for the community.

Other ways to generate reference panel samples are also possible. For example, the computing server 130 may collect a set of samples and gradually filter and refine the samples until high-quality reference panel samples are selected. For example, a candidate reference panel sample may be selected from an individual whose recent ancestors were born at a certain birthplace. The computing server 130 may also draw sequence data from the Human Genome Diversity Project (HGDP). Various candidates may be manually screened based on their family trees, relatives' birth location, and other quality controls. Principal component analysis may be used to create clusters of genetic data of the candidates. Each cluster may represent an ethnicity. The predictions of the ethnicity of those candidates may be compared to the ethnicity information provided by the candidates to perform further screening.

The ethnicity estimation engine 245 estimates the ethnicity composition of an inheritance dataset of a target individual. The inheritance datasets used by the ethnicity estimation engine 245 may be genotype datasets or haplotype datasets. For example, the ethnicity estimation engine 245 estimates the ancestral origins (e.g., ethnicity) based on the individual's genotypes or haplotypes at the SNP sites. To take a simple example of three ancestral populations corresponding to African, European and Native American, an admixed user may have nonzero estimated ethnicity proportions for all three ancestral populations, with an estimate such as [0.05, 0.65, 0.30], indicating that the user's genome is 5% attributable to African ancestry, 65% attributable to European ancestry and 30% attributable to Native American ancestry. The ethnicity estimation engine 245 generates the ethnic composition estimate and stores the estimated ethnicities in a data store of computing server 130 with a pointer in association with a particular user.

In some embodiments, the ethnicity estimation engine 245 divides a target inheritance dataset into a plurality of windows (e.g., about 1000 windows). Each window includes a small number of SNPs (e.g., 300 SNPs). The ethnicity estimation engine 245 may use a directed acyclic graph model to determine the ethnic composition of the target inheritance dataset. The directed acyclic graph may represent a trellis of an inter-window hidden Markov model (HMM). The graph includes a sequence of a plurality of node groups. Each node group, representing a window, includes a plurality of nodes. The nodes represent different possibilities of labels of genetic communities (e.g., ethnicities) for the window. A node may be labeled with one or more ethnic labels. For example, a level includes a first node with a first label representing the likelihood that the window of SNP sites belongs to a first ethnicity and a second node with a second label representing the likelihood that the window of SNPs belongs to a second ethnicity. Each level includes multiple nodes so that there are many possible paths to traverse the directed acyclic graph.

The nodes and edges in the directed acyclic graph may be associated with different emission probabilities and transition probabilities. An emission probability associated with a node represents the likelihood that the window belongs to the ethnicity labeling the node given the observation of SNPs in the window. The ethnicity estimation engine 245 determines the emission probabilities by comparing SNPs in the window corresponding to the target inheritance dataset to corresponding SNPs in the windows in various reference panel samples of different genetic communities stored in the reference panel sample store 240. The transition probability between two nodes represents the likelihood of transition from one node to another across two levels. The ethnicity estimation engine 245 determines a statistically likely path, such as the most probable path or a probable path that is at least more likely than 95% of other possible paths, based on the transition probabilities and the emission probabilities. A suitable dynamic programming algorithm such as the Viterbi algorithm or the forward-backward algorithm may be used to determine the path. After the path is determined, the ethnicity estimation engine 245 determines the ethnic composition of the target inheritance dataset by determining the label compositions of the nodes that are included in the determined path. U.S. Pat. No. 10,558,930, entitled “Local Genetic Ethnicity Determination System,” granted on Feb. 11, 2020, and U.S. Pat. No. 10,692,587, granted on Jun. 23, 2020, entitled “Global Ancestry Determination System” describe different example embodiments of ethnicity estimation.

The tree management engine 250 performs computations and other processes related to users' management of their data trees such as family trees. The tree management engine 250 may allow a user to build a data tree from scratch or to link the user to existing data trees. In some embodiments, the tree management engine 250 may suggest a connection between a target individual and a family tree that exists in the family tree database by identifying potential family trees for the target individual and identifying one or more most probable positions in a potential family tree. A user (target individual) may wish to identify family trees to which he or she may potentially belong. Linking a user to a family tree or building a family may be performed automatically, manually, or using techniques with a combination of both. In an embodiment of an automatic tree matching, the tree management engine 250 may receive an inheritance dataset from the target individual as input and search related individuals that are IBD-related to the target individual. The tree management engine 250 may identify common ancestors. Each common ancestor may be common to the target individual and one of the related individuals. The tree management engine 250 may in turn output potential family trees to which the target individual may belong by retrieving family trees that include a common ancestor and an individual who is IBD-related to the target individual. The tree management engine 250 may further identify one or more probable positions in one of the potential family trees based on information associated with matched genetic data between the target individual and those in the potential family trees through one or more machine learning models or other heuristic algorithms. For example, the tree management engine 250 may try putting the target individual in various possible locations in the family tree and determine the highest probability position(s) based on the inheritance dataset of the target individual and inheritance datasets available for others in the family tree and based on genealogy data available to the tree management engine 250. The tree management engine 250 may provide one or more family trees from which the target individual may select. For a suggested family tree, the tree management engine 250 may also provide information on how the target individual is related to other individuals in the tree. In a manual tree building, a user may browse through public family trees and public individual entries in the genealogy data store 200 and individual profile store 210 to look for potential relatives that can be added to the user's family tree. The tree management engine 250 may automatically search, rank, and suggest individuals for the user conduct manual reviews as the user makes progress in the front-end interface 260 in building the family tree.

As used herein, “pedigree” and “family tree” may be interchangeable and may refer to a family tree chart or pedigree chart that shows, diagrammatically, family information, such as family history information, including parentage, offspring, spouses, siblings, or otherwise for any suitable number of generations and/or people, and/or data pertaining to persons represented in the chart. U.S. Pat. No. 11,429,615, entitled “Linking Individual Datasets to a Database,” granted on Aug. 30, 2022, describes example embodiments of how an individual may be linked to existing family trees.

The front-end interface 260 may render a front-end platform that displays various results determined by the computing server 130. The platform may take the form of a genealogy research and family tree building platform and/or a personal DNA data analysis platform. The platform may also serve as a social networking system that allows users and connect and build family trees and research family relations together. The results and data may include the IBD affinity between a user and another individual, the community assignment of the user, the ethnicity estimation of the user, phenotype prediction and evaluation, genealogy data search, family tree and pedigree, relative profile and other information. The front-end interface 260 may allow users to manage their profile and data trees (e.g., family trees). The users may view various public family trees stored in the computing server 130 and search for individuals and their genealogy data via the front-end interface 260. The computing server 130 may suggest or allow the user to manually review and select potentially related individuals (e.g., relatives, ancestors, close family members) to add to the user's data tree. The front-end interface 260 may be a graphical user interface (GUI) that displays various information and graphical elements.

The front-end interface 260 may take different forms. In one case, the front-end interface 260 may be a software application that can be displayed on an electronic device such as a computer or a smartphone. The software application may be developed by the entity controlling the computing server 130 and be downloaded and installed on the client device 110. In another case, the front-end interface 260 may take the form of a webpage interface of the computing server 130 that allows users to access their family tree and genetic analysis results through web browsers. In yet another case, the front-end interface 260 may provide an application program interface (API). In some embodiments, the front-end interface 260 may be rendered as part of the content in an extended reality device, such as a head-mounted display or a phone camera that is integrated with augmented reality features.

The front-end interface 260 may provide various front-end visualization features. In some embodiments, a family tree viewer may render family tree built by users and/or managed by the tree management engine 250. The family tree may be displayed in a nested nodes and edges connected based on family relationships or genetic matches determined by various genetic data analysis engines discussed in FIG. 2. The family trees may include attached records that are part of records in the genealogy data store 200, including records that are uploaded by users and gallery images. The user may assign a focal person to a family tree and the family tree is displayed with the focus (such as positioning the focal person at the center or relative prominent position of the tree) around the focal person. A user may change the focal person and the family tree may shift accordingly based on the relationships and relative positions of members in the family tree. Each person in the family tree may be associated with historical photos from gallery images, historical genealogy records such as life event records, one or more stories and live events associated with the person, and metadata such as family relationships and other family trees associated with the person.

In some embodiments, visualization features provided by the front-end interface 260 may include a map feature. A map may be a geographical map that may take the form of a digital map, a historical physical map, and/or a historical map overlaid on a digital map. A user may select a geographical location and the front-end interface 260 displays relevant genealogical or genetic records associated with the location, such as an ancestor's lifetime events, birth locations of DNA matches, mitigation patterns of ancestors across different locations over time and associated genealogical records, residence maps that provide specific locations of historical persons' events, and historical maps overlaying on a digital map to contextualize ancestors' records and events. The map feature may also provide interactive features to allow users to view historical documents, photographs, and stores associated with the geographical locations. The map feature may also allow users to adjust timeframes, displaying changes in locations and migrations over different periods.

In some embodiments, visualization features provided by the front-end interface 260 may include a story feature that provides multimedia narratives about a person, such as the person's live events and family history. The story feature allows a user to compile various graphical and genealogical elements such as photos, documents, historical records, and personal anecdotes into a timeline to summarize a narrative. The story may be arranged in an appropriate spatial manner such as a linear arrangement that arranges various graphical elements based on the creator's selection.

### Example Process for Determining Inheritance Labels of Users

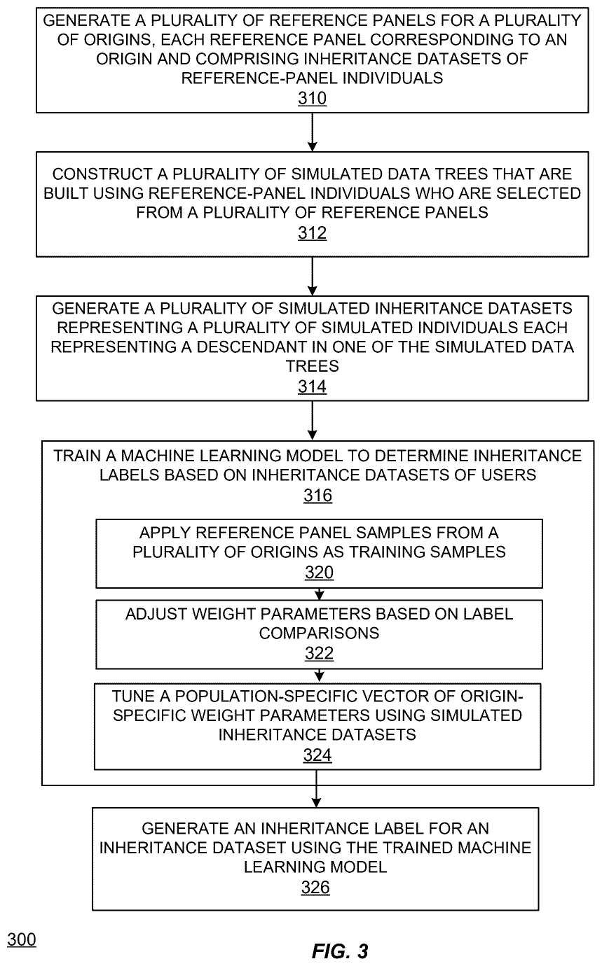

FIG. 3 is a flowchart depicting an example process 300 for determining inheritance labels of users based on inheritance datasets of individuals. The process 300 may be performed by one or more engines of the computing server 130 illustrated in FIG. 2, such as the ethnicity estimation engine 245. The process 300 may be embodied as a software algorithm that may be stored as computer instructions that are executable by one or more processors. The instructions, when executed by the processors, cause the processors to perform various steps in the process 300. In various embodiments, the process may include additional, fewer, or different steps. While various steps in process 300 may be performed with the use of computing server 130, each step may be performed by a different computing device.

The process 300 for determining inheritance labels of users based on inheritance datasets of users advantageously is configured to address one or more limitations of existing approaches for determining inheritance labels of users based on inheritance datasets of users, such as existing approaches being hard to explain or understand, time consuming and processing resource-intensive, optimized on a small set of an inheritance dataset database, difficult if not impossible to recreate, expensive due to high number of computer steps, repetitive with small incremental value, and ill-suited to adding new regions in domains representing a high degree of admixture in the inheritance dataset database. The process 300 for determining inheritance labels of users based on inheritance datasets of users according to embodiments of the disclosure advantageously is easier to automate and understand, is inheritance-dataset focused, reduces the number of compute and manual steps, can be recreated automatically at any desired cadence, and can add new regions in previously ambiguated regions.

In this disclosure, particularly in FIG. 3 through FIG. 7C, named entities may refer to individuals. Data reference panels or simply “reference panels” may be or comprise inheritance datasets of individuals who are representative of a data-inheritance origin. Data-inheritance origins may be referred to more simply herein as “origins” and describe how data may be inherited from real-world events. Data may be inherited and may evolve based on real-world events that are not always recorded or documented. Yet, while the real-world events may not be completely documented, the change and inheritance of those events may be traceable by comparing data strings of, among, and/or between data instances. For example, two data instances may be generated or exist independently and individually reflect the status of their respective named entities or events. The data patterns in the data instances may reflect the natures, histories, or characteristics of data-inheritance sources such as related or unrelated named entities or events. However, multiple data instances or corresponding named entities or events may be inherited at least in part from one or more common sources so that the data instances share some similarities in the data patterns. As such, the nature of inheritance may be revealed by analyzing and comparing the multiple data instances, and sometimes a large number of data instances. Those real-life events that result in shared data strings among data instances may be referred to as data-inheritance events, even though those real-life events, at the time of the occurrence, may not involve data or data generation at all. For example, the real-life events may be historical events that occurred before the invention of computer or data, but present data instances may still reflect those historical events.

In some cases, however, only portions of data strings are inherited from a data-inheritance event and the precise locations and extent of inheritance are not apparent without a complex process to analyze and compare the one or more data instances. In some cases, for a given data instance, it may be difficult to identify how various portions of the data instance are inherited from different real-world events or named entities. A data instance may inherit data from various sources that are referred to as inheritance sources. Various processes described herein provide solutions to identify inheritance sources of a data instance by analyzing the data patterns in other data instances and identifying how data are passed down.

In some embodiments, the computing server 130 generates a plurality of reference panels for a plurality of origins (step 310). Each reference panel may correspond to an origin. Each reference panel may include inheritance datasets of reference-panel individuals representative of the origin. At least one of the reference-panel individuals of a particular origin may be selected based on a number of individuals in the particular origin to whom the inheritance dataset of the reference-panel individual are matched.

An origin may refer to a geographic, ethnic, or ancestral entity derived from genetics or genealogy. When creating reference panels for genetic comparisons, the origin may form the basis for categorization. For example, the origin may correspond to an ethnicity region, an ethnicity subregion, or an ethnicity categorization within a subregion. The computing server 130 may define the granularity of origins based on data available to the computing server 130 and manually defined criteria such as known geographic regions and historical migration knowledge. For example, with respect to Europe, examples of regions may include Northern Europe, Western Europe, Eastern Europe. In one embodiment, an origin may refer to any one of these regions. For instance, the Northern Europe origin may capture the unique genetic variants more common in the corresponding Northern European population.

Within Northern Europe, there may be further subdivisions or subregions such as Scandinavia, British Isles, and Baltic States. Thus, in some embodiments, an origin may refer to a subregion. A subregion may be split into more specific subregions. For example, Scandinavia may be split into Sweden and Denmark. Thus, in some embodiments, an origin may refer to a specific subregion, e.g. an ethnicity subregion. A subregion may be split into even more precise geographic categories. For instance, Sweden may be subdivided into Southern Sweden (Götaland), Central Sweden (Svealand), and Northern Sweden (Norrland). Thus, in some embodiments, an origin may refer to a precise geographic category within a subregion.

An inheritance dataset may correspond to a genetic dataset. For example, an inheritance dataset of an individual may be a digital record of the individual's genetic information, including nucleotide data such as SNPs, full sequences of genetic markers, genotyping data, or entire genomic sequences. This information may be used, for example, to examine the individual's genetic history or identify genetic variants. Further example of inheritance dataset is discussed in FIG. 2 with respect to the genetic data store 205.

In some embodiments, the computing server 130 may generate a plurality of reference panels for a plurality of corresponding origins by collecting inheritance datasets, categorizing each collected dataset based on geographic area, ethnic group, or a genetic pool associated therewith, and creating reference panels based on the categorized datasets. That is, the computing server 130 may receive inheritance datasets for various individuals from the reference panel sample store 240 or the genetic data store 205. These individuals may be from or associated with diverse geographical regions or ethnic backgrounds. Each collected dataset may be associated with an origin, which may represent a geographic area, ethnic group, or a genetic pool. The origins with which the collected datasets are associated may be an intrinsic association or may be ascertained via preexisting metadata, in embodiments. In other embodiments, the origins with which the collected datasets are associated may be determined by the computing server 130.

For each origin, the computing server 130 may create a reference panel that includes the collected genetic datasets from individuals of that specific origin. That is, within or for each origin, certain individuals may be selected as reference-panel individuals. The selection of these individuals may be based on the number of individuals from the same origin or genetic community to whom their inheritance datasets show a match. A match may be an IBD match or any other genetic match. These selected individuals may have representative genetic characteristics of their respective origins, facilitating downstream comparisons thereagainst for inheritance-related determinations.

In some embodiments, the computing server 130 may select at least one of the reference-panel individuals of the particular origin by: filtering a plurality of candidates based on inheritance labels, such as, but not limited to, pre-existing inheritance labels; identifying, for each candidate, a number of the individuals in or associated with the particular origin whose inheritance datasets match the inheritance dataset of the candidate; and selecting a candidate as one of the reference-panel individuals based on the number of matched individuals that correspond to the candidate compared to numbers of matched individuals of other candidates and/or other origins.

For example, to generate a candidate pool for a particular origin or, in other words, a plurality of candidate reference-panel datasets, the computing server 130 may identify the respective inheritance labels associated with that particular origin. These inheritance labels may be indicative of the genetic characteristics or markers that are significantly associated or prevalent within the members of that particular origin. The computing server 130 may access and/or scan through a database of genetic datasets to select individuals that possess these inheritance labels in their genetic information. Each individual with a relevant inheritance label may be deemed a candidate and may be included in the pool. Using this approach, the computing server 130 may generate a candidate pool that includes a plurality of candidates having inheritance labels related to the particular origin. In embodiments, the inheritance labels are pre-existing in a database, such as the genetic data store 205. In other embodiments, the computing server 130 may access a plurality of genetic or inheritance datasets that do not yet have corresponding metadata indicating an inheritance label; in such embodiments, the computing server 130 may utilize a suitable modality, such as the ethnicity estimation engine 245 using existing reference panel(s), to determine inheritance labels for the plurality of accessed inheritance datasets, and then identify such inheritance datasets as have been determined to correspond to a particular inheritance label as candidates.

The computing server 130 may identify, for each candidate of the candidate pool, a number of the individuals in the particular origin whose inheritance datasets match the inheritance dataset of the candidate. For example, the computing server 130 may compare the inheritance datasets of the individuals in the specified origin to the inheritance dataset of the candidate. This may include comparing IBD segments of the candidate and the individuals in the specified origin to find matched (or shared) segments. This may also include any other genetic match between the candidate and the individuals in the specified origin.

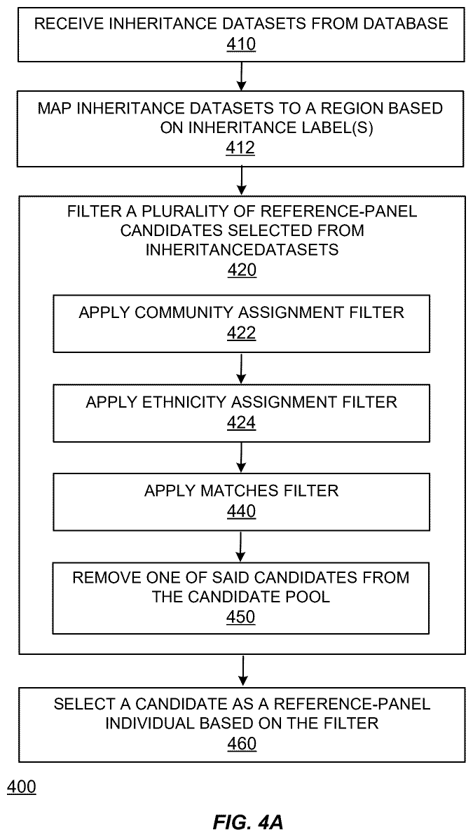

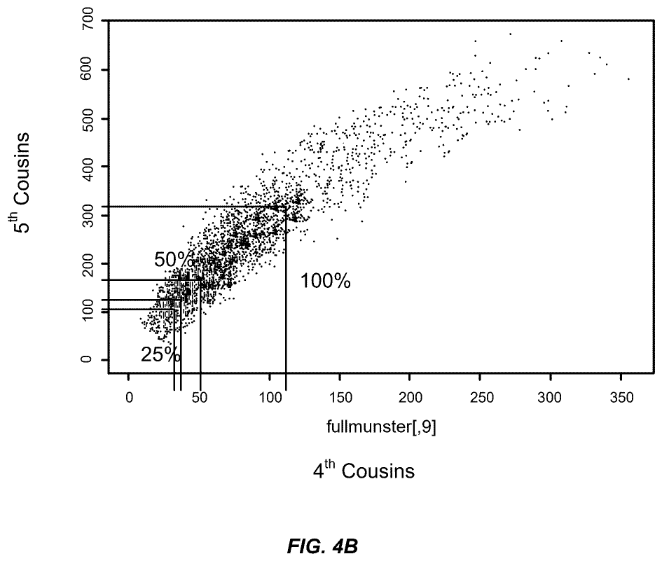

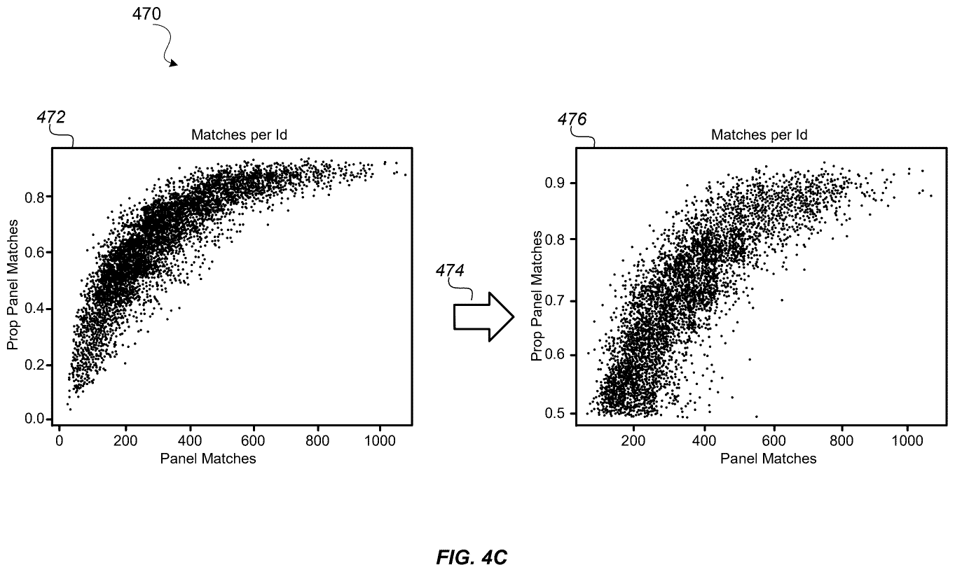

In various embodiments, one or more reference-panel individuals may be generated using the processes discussed in FIG. 2 in association with the reference panel sample store 240. Alternatively, or additionally, one or more reference-panel individuals may be generated using a process described in the paragraph above, which will be further discussed in process 400 in association with FIG. 4A.

Continuing with reference to FIG. 3, in some embodiments, the computing server 130 constructs a plurality of simulated data trees using reference-panel individuals (step 312). The individuals may be selected from the plurality of reference panels. Each of these simulated data trees may be associated with a particular geographical location, which may have a unique demographic profile. The computing server 130 may retrieve, access, or determine the geographical location's population composition and sample reference-panel individuals from multiple panels according to the population composition. The sampled reference-panel individuals may be positioned within pertinent nodes of the simulated data tree. Their placements may be guided by the generation-specific population composition.

In some embodiments, the computing server 130 may construct a particular simulated data tree by retrieving, accessing, or determining the population composition of the particular geographical location, sampling the reference-panel individuals from the plurality of reference panels, and representing the sampled reference-panel individuals in the nodes of the particular simulated data tree based on the population composition of the particular geographical location.

Retrieving, accessing, or determining the population composition of the particular geographical location may be one of the first steps in constructing the simulated data tree. The population composition may refer to the particular mix or proportions of individual genetic profiles from a specific geographical area. The population composition may include information related to percentages of individuals with the plurality of origins. The population composition may also include generation-specific compositions. Accessing the population composition of the particular geographical location may include accessing the genetic data store 205 to retrieve data about individuals' community, ethnicity, genetic variants, and/or other relevant genetic information. One of the purposes of this step is to create a representative sample that mirrors the genetic diversity of the particular geographical location. For example, if the particular geographical location is England, the computing server 130 may access the genetic data store 205 to retrieve genetic profiles unique to and/or associated with that region. The retrieved data may be used to sample reference-panel individuals who may be represented in the nodes of a simulated data tree, which is tailored for that specific geographical location.

The simulated data tree may be a hypothetical family tree. The simulated data tree may be generated, using statistical probabilities, to model a population composition of the particular geographical location which the simulated data tree tries to simulate. The simulated family individuals in the data tree may be selected from a pool of representative reference panels based on the population composition. For example, in a particular geographical location, the population composition may be determined to be 50% from a first origin and 50% from a second origin. In turn, the computing server 130 may generate a simulated data tree that draws approximately 50% from a first reference panel corresponding to the first origin and 50% from a second reference panel corresponding to the second origin.

In some embodiments, the simulation may also be generation specific. The simulated hypothetical family tree may comprise a plurality of generations relative to a bottommost or “descendant” generation up to a topmost progenitor generation, with suitable numbers of ancestors in intervening generations. For example, a “grandparent generation” may be a generation two generations up from the bottommost descendant generation such that the four persons represented therein represent the descendant's grandparents. Thus, for example, in a grandparent generation, the population data may indicate that, instead of a 50%-50% split, the population composition is 70% from the first origin and 30% from the second origin. In turn, the computing server 130, for the grandparent generation in the simulated data tree, may draw appropriately 70% from the first reference panel and 30% from the second reference panel. This may be performed for the composition at each generation of a simulated data tree. Suitable approximations, corrections, or other operations may be performed as suitable for particular generations of a tree. For example, for a grandparent generation which inherently comprises four persons, a 70/30 split between origins may entail rounding to the nearest division that lends itself to a four-way split, with three of the grandparents selected from the first origin and one grandparent selected from the second origin. Any suitable operations for any suitable population composition and generation may be performed.

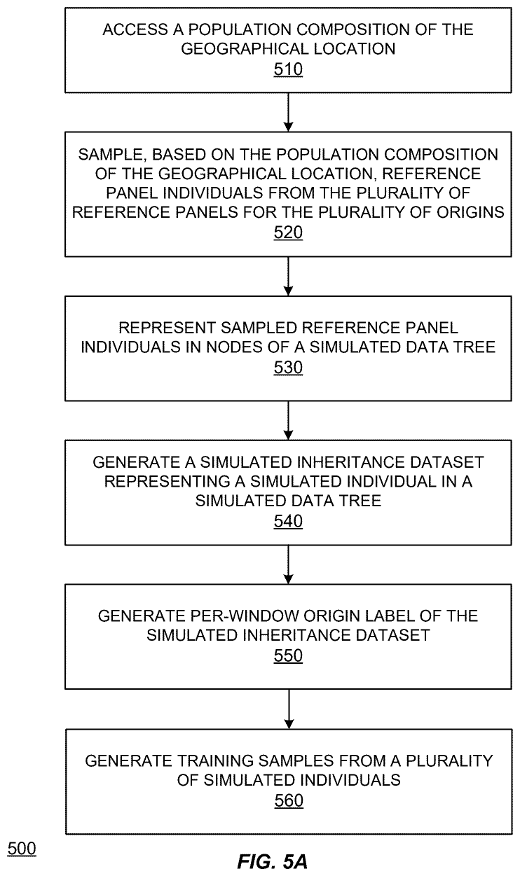

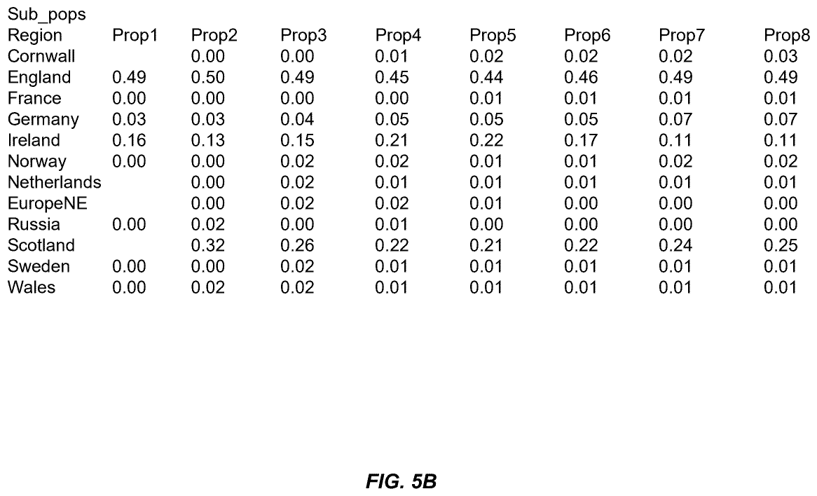

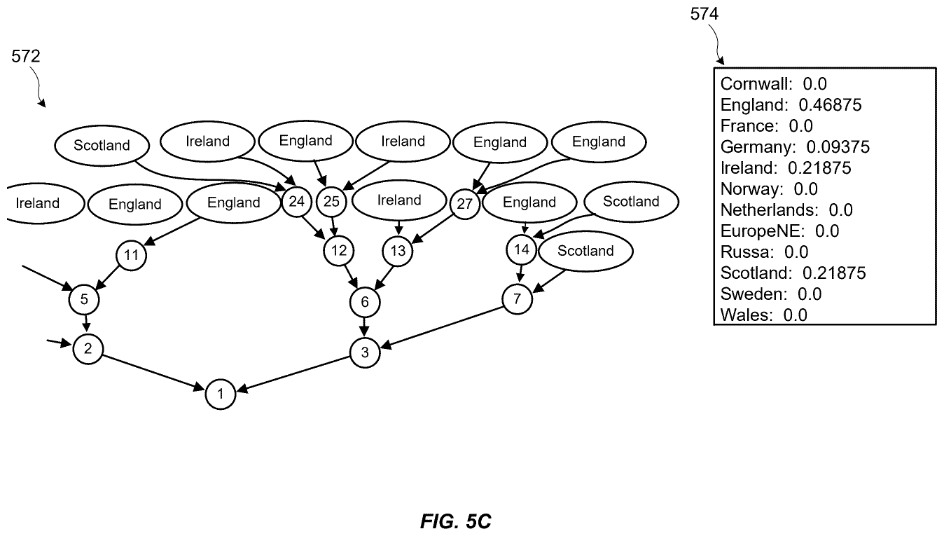

Further detail and examples related to generating one or more simulated data tree are discussed in FIG. 5 in association with the process 500.

Continuing with reference to FIG. 3, in some embodiments, the computing server 130 may generate a plurality of simulated inheritance datasets representing a plurality of simulated individuals each representing a descendant in one of the simulated data trees (step 314). The process of generating the simulated inheritance datasets may include simulating a descendant of the reference-panel individuals who are placed in the particular simulated data tree to generate an inheritance dataset of the descendant (e.g., a simulated genotype or a simulated genetic dataset of the descendant), simulating a plurality of inheritance events such as reproduction events based on how the family tree is simulated (e.g., a simulation of a reproduction event between a first reference-panel individual situated in the family tree as a father and a second reference-panel individual situated as a mother), generating the particular simulated inheritance dataset of the particular simulated descendant based on the plurality of inheritance events, and repeating the process to generate additional descendants for, e.g., additional or subsequent generations and/or for additional simulated data trees. The simulated descendant's inheritance datasets may be generated based on a number of reproduction events such as between, e.g., great great grandparents, great grandparents, grandparents, and parents, depending on the number of generations in the simulated family tree.

In some embodiments, the simulated data trees may include three, four, five, six, seven, eight, or more generations as suitable, with simulated reproduction events for each pertinent generation. The reproduction events may involve, e.g., simulation of meiosis and recombination events based on, e.g., linkage disequilibrium. The generation of a plurality of simulated inheritance datasets may be similar to a Monte Carlo simulation. For example, for a particular simulated data tree, different inheritance datasets of different descendants may be simulated with the same combinations of reference-panel individuals utilized in the simulated data tree as simulated relatives by using different meiosis events and recombination events. Additionally, or alternatively, a number of different simulated data trees may be generated using the same population composition by randomly drawing different reference-panel individuals from the corresponding reference panels based on the population composition. In turn, each simulated data tree may be used to generate one or more simulated descendants. The simulation may entail the use of any suitable modality, such as a machine learned or other algorithm, for simulating reproduction events among and between the selected inheritance datasets.

The simulated inheritance datasets of descendants may be processed by the computing server 130 according to or using one or more utilities such as the ethnicity estimation engine 245 to obtain inheritance labels for the simulated inheritance datasets. The determined inheritance labels may be used by the computing server 130 for comparison against results obtained from other inheritance datasets, e.g. real inheritance datasets, and thereby to improve the results obtained for the other inheritance datasets by training, tuning, or otherwise modifying the use of a utility or modality for determining inheritance datasets.

It was surprisingly found that the simulation process significantly improves the quality of samples that are based on the simulated inheritance datasets. The process may begin by selecting a descendant position in the simulated data tree. The simulated individual's genetics may be based on those of a real-world population represented by the above-mentioned reference-panel individuals. The simulation process is able to make realistic assumptions about the simulated individual's genetic makeup based on the known genetic profiles of the population represented by the reference panels. For example, if a simulated person is being generated for a simulated data tree modeling genetic diversity in a specific geographical region, particularly an admixed region, such as Mexico, then individuals from various origins that represent the genetic diversity of that region may be used. Hence, multiple reference panels from different origins that represent or correspond to the admixed nature of the geographical region may be utilized or relied upon in the simulation. Generating a simulated individual as a descendant of these reference-panel individuals may ensure that the genetic traits attributed to those reference-panel individuals are in line with those common in the admixed population. Therefore, this step may provide realistic portrayals of genetic distribution and variation in the simulated data tree. It may also allow a machine learning model used in conjunction with data from the simulated data trees to account for the real-world complexity and variety of genetic inheritance tied to a certain geographical or demographic background. That is, by using the simulated inheritance datasets to tune a vector of population- or origin-specific weight parameters for augmenting a machine learning model, the machine learning model's ability to accurately predict inheritance labels for new inheritance datasets is markedly improved as is its ability to generate and generation accurate predictions for new reference panels and corresponding regions.

Further detail and examples related to generating one or more simulated inheritance datasets of simulated descendants are discussed in regard to FIG. 5 in association with the process 500.

Continuing with reference to FIG. 3, in some embodiments, the computing server 130 trains a machine learning model that is configured to determine inheritance labels of users based on inheritance datasets of the users (step 316). The inheritance labels may be identifiers within or determined based on an individual's inheritance dataset, e.g. genetic dataset, that represent specific attributes or characteristics. For example, the inheritance labels may represent biological or demographic information derived from a person's genetic data. In some embodiments, the inheritance labels may include ethnicity labels, community labels, geographic origin labels, and genetic trait labels. The ethnicity labels may indicate the ethnic groups that an individual's genetic data most likely align with, based on their genomic sequence or specific genetic markers.

The community labels may indicate genetic affiliations to certain communities or groups of a more-recent and/or more-granular nature than ethnic extractions. The communities may be associated with a geographical location, a common ethnicity, or specific genetic traits. The geographic origin labels may associate an individual's genetic data to specific geographical regions, suggesting where their ancestors might have originated from. The genetic trait labels may indicate the presence of certain genetic traits, like curly hair, lactose tolerance, etc., based on the individual's genetic data.

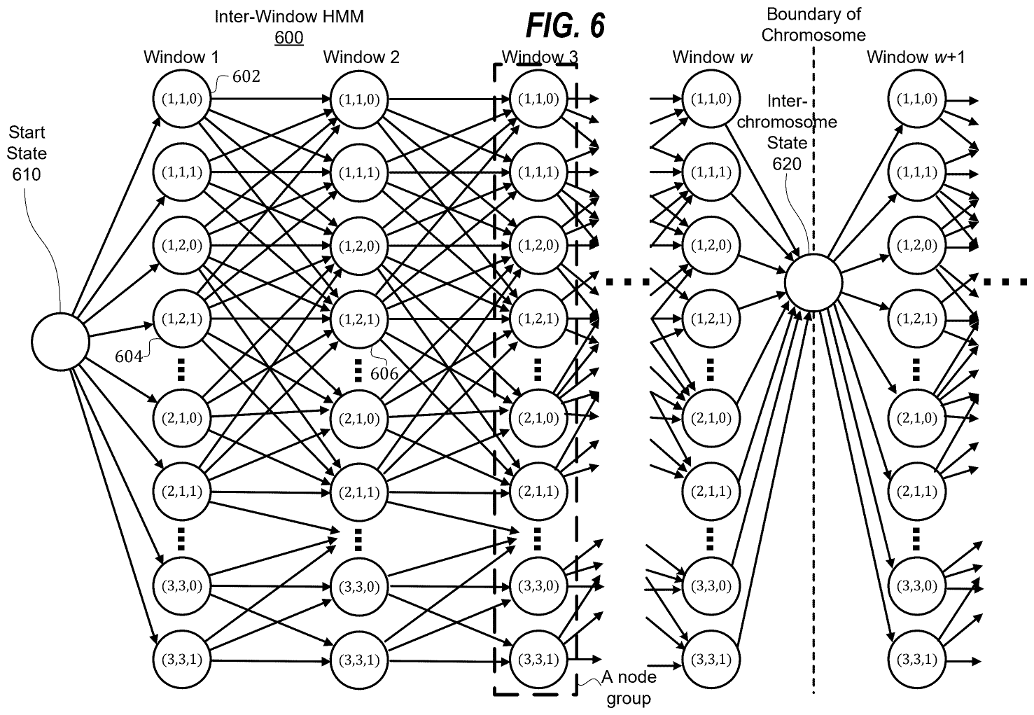

Each inheritance label may correspond to a particular genomic segment (e.g., a first genomic segment appears to be inherited from a French ethnicity or ancestor, a second genomic segment appears to be inherited from a Japanese ethnicity or ancestor, etc.). The genomic segments may be phased (separating the genomic segments from first parent and the second parent) or unphased. Using the machine learning model, the final determination of ethnicity-specific inheritance labels for a user may be something such as 60% French, 30% Japanese, and 10% Chinese, etc. In some embodiments, the machine learning model may be an HMM with windows that represent segments of inheritance data, e.g. genomic segments. An example of an HMM is provided in FIG. 6.

Training the machine learning model may include applying reference panel samples from a plurality of origins as training samples (step 320), applying the machine learning model to predict inheritance labels of the training samples and adjusting weight parameters of the machine learning model based on comparisons between the predicted inheritance labels and the inheritance labels associated with the reference panel samples (step 322), and tuning a population- or origin-specific vector of weights or weight parameters using simulated inheritance datasets (step 324). During inference, the trained machine learning model may be used to generate an inheritance label for an inheritance dataset for a user (step 326). Further details on the origin-specific weight parameters are described regarding FIG. 6.

The computing server 130 may initiate origin-specific weight parameters in the machine learning model at the outset of or separate from training using reference panel samples. The origin-specific weight parameters may correspond to the level of impact that a specific population may have on the outcomes of the machine learning model and may be additional to known and previously used weights and variables for inheritance-label determination. For example, each population may be assigned an origin-specific weight parameter. One of the purposes of the origin-specific weight parameters is to optimize the machine learning model's overall performance. The origin-specific weight parameters may be used to adjust the impact of specific populations based on how well they match real-world data. In situations where the machine learning model is over-predicting or under-predicting the representation of a certain population based on known ground-truth data, the origin-specific weight parameters of that population may be adjusted accordingly. The initial origin-specific weight parameters may be randomly selected, may all be uniform, or may be based on some other metric or estimate as suitable.

For example, if the machine learning model predicts more instances of a certain genetic marker associated with a population than what is observed in real-world data, the origin-specific weight parameter for that population may be correspondingly adjusted, e.g., by being reduced. If the machine learning model under-predicts the instances of a genetic marker associated with a different population, its origin-specific weight parameter may be correspondingly adjusted, e.g., by being increased. The weight adjustments may improve the accuracy, precision, and recall metrics of the machine learning model. For example, the process may iteratively tune the origin-specific weight parameters until the machine learning model's performance meets the established benchmarks.

Each origin may be assigned a weight parameter in accordance with its predicted impacts on the machine learning model's outcomes. This assignment may be based on various factors such as the initial volume of training data associated with each origin or prior knowledge about the impacts of different origins. As the machine learning model is trained using the simulated inheritance datasets, the computing server 130 may adjust the origin-specific weight parameters through the training process and/or based on the model's performance in predicting the inheritance labels for training samples.

The origin-specific weight parameters for the origins may be provided to and utilized with the machine learning model as a vector of tunable per-population weights, with, in embodiments, a single value per population or origin. The vector may be global and not be dependent on state of the machine learning model. In other embodiments, the origin-specific weight parameters are multiplied against each state's probabilities. An example of such a vector is shown below in Table 1.

TABLE 1

| Ethnicity | Origin-specific weight parameter |
|---|---|
| Balkans | 1.001 |
| Baltic | 0.972 |
| Basque | 0.981 |
| Cornwall | 0.954 |
| Cyprus | 0.949 |
| Denmark | 0.982 |
| England | 1.045 |
| European Jew | 1.078 |
| Europe Northeast | 1.12 |
| Finland | 0.954 |
| France | 1.055 |
| Germany | 1.024 |

The vector may operate to increase or decrease origin-specific probabilities. It was surprisingly found that this advantageously reduces the computation requirements of training the machine learning model by numerous orders of magnitude, as the number of runs or epochs of the machine learning model to reach convergence may be reduced from hundreds of thousands, per previous approaches, to as few as five, while improving performance of the machine learning model based on, e.g., overlap between expected and observed inheritance-label assignments.

It will be appreciated that in embodiments, the vector of tunable per-population weights with a single value per population or origin, the vector being in embodiments independent of state, may be determined according to a process such as the process described below, and may be independent of a training process for the machine learning model. Indeed, the vector of tunable per-population weights may be determined separately using, e.g., simulated datasets generated according to the process described regarding and depicted in FIG. 5B.

It has been surprisingly found that generating simulated datasets for generating a vector of tunable per-population weights as described herein advantageously facilitates testing, training, and utilizing weight vectors yielding far more accurate results and the ability to add far more regions to a reference panel than has been possible using existing approaches. That is, the vector of tunable per-population weights, tuned using simulated datasets, allow for enormous cost reductions from reduced processing requirements as orders of magnitude fewer iterations are needed to arrive at accurate inheritance-label assignments while enabling the addition of new regions where none had existed before given, in some instances, a paucity of available data.

The computing server 130 may initiate a machine learning model structure that includes nodes, windows, and parameters. A window may correspond to a genomic segment, phased or unphased. For example, each inheritance dataset or components thereof, such as chromosomes of a genotype, may be divided into a plurality of predefined windows. Each window may include a plurality of nodes. Each node may represent an origin. Predictions regarding inheritance labels for the windows may be associated with or based on weights of the plurality of nodes. Further detail of the structure of various examples of the machine learning model is discussed in FIG. 2 in association with the ethnicity estimation engine 245.

The computing server 130 may apply the plurality of reference panel sample datasets as training samples. In embodiments, the training samples may include both reference panel sample datasets and simulated datasets as obtained as described herein. In some embodiments, only reference panel samples are used as training samples. The training samples may include admixed individuals that are simulated from plurality of simulated data trees and/or non-admixed individuals that are sampled from actual user datasets and/or reference panels. For example, admixed training samples may be obtained using the process 500 described in FIG. 5 while non-admixed training samples may be obtained from the reference panel sample store 240. The computing server 130 may apply the machine learning model to predict inheritance labels of the training samples. Each of the training samples may be associated with corresponding inheritance labels as ground truth. For example, in embodiments where simulated datasets are included as training samples, the labels generated for the simulated inheritance datasets as described herein and/or determined based on the construction or contents of the simulated data tree. The machine learning model may process the training samples to learn underlying patterns and correlations between inheritance datasets and their corresponding inheritance labels and to adjust weights or other parameters of the machine learning model accordingly. The machine learning model may use what it has learned to predict inheritance labels of new, unseen data.

The computing server 130 may compare the predicted inheritance labels to actual labels obtained from the plurality of simulated data trees. The process whereby the computing server 130 compares predicted inheritance labels to actual labels obtained from the simulated data trees primarily may provide a measure of how accurately the machine learning model has performed. Upon the machine learning model predicting the inheritance labels for the test samples, the machine learning model may output a set of predicted inheritance labels. The computing server 130 may access the corresponding actual labels from the simulated data trees that were used in model training. These actual labels may serve as the ground truth against which predictions are validated. The actual labels may indicate the true geographic origin, ethnic group, or ancestral pool, while the predicted labels may represent the model's inferential output for the test samples. The machine learning model's prediction accuracy may be validated against actual labels derived from simulated data trees to test its reliability and effectiveness. The accuracy of the predictions may be assessed and refined over time to provide ongoing improvement and optimization of the machine learning model.

In embodiments, the training samples, which may include reference panel samples, are divided into subsets, such as that a different set of samples is used to train the machine learning model, a different set of samples is used to train the weight vectors, and a different set of samples is used to validate the weight vectors and/or the machine learning model. That is, the training samples may be split into groups for training, testing vis simulation, and validation vis simulation. In embodiments, the set of samples used to train the weight vectors includes the training samples with which the simulated data trees are populated based on population composition. By keeping these training samples separate from the training samples and validation samples, overfitting and other issues are avoided. Any suitable discretization of the training samples (e.g., 60/20/20) may be utilized.

In embodiments where simulated datasets are used in training the machine learning model, the actual labels obtained from the plurality of simulated data trees may be generated by dividing a particular simulated inheritance dataset in a particular training sample into a plurality of windows (which may correspond to the windows into which the reference panel samples and input datasets are divided), examining how a segment of the particular simulated inheritance dataset in a particular window is inherited in a particular simulated data tree, identifying a reference-panel individual in the particular data tree who passes down the identified segment to the simulated inheritance dataset, determining an origin label of said reference-panel individual, and using the origin label as the actual label for the particular window. This may be performed for all or substantially all of the windows of the simulated dataset.

The generation of the actual labels may begin by dividing a particular simulated inheritance dataset in a training sample into numerous windows. These windows may provide for a more granular examination of the dataset. The second step may include determining how a genetic segment within a particular window is inherited in the corresponding simulated data tree. This genetic segment may correspond to a sequence of genetic information within the inheritance dataset. The third step may include identifying the reference-panel individual from the simulated data tree. The reference-panel individual may be someone who passes down the genetic segment to the simulated inheritance dataset and may be the genetic source of that segment. In the fourth step, the computing server 130 may determine the origin label of this identified reference-panel individual. The origin label may correspond to the geographical, ethnic, or ancestral group to which the individual belongs. In a further step, the computing server 130 may use the determined origin label as the actual label for the inheritance dataset. This provides that actual label as being representative of the original ancestor from which the genetic segment was inherited.

The aggregated labels, and corresponding proportions of labels assigned to windows, may be utilized to determine an actual proportion or percentage of inheritance assignment(s) or label(s) for the simulated inheritance dataset. For example, if an inheritance dataset is divided into, say, 100 windows, and 25 of those windows are determined to correspond to an English ethnicity, 25 of those windows are determined to correspond to a Japanese ethnicity, and 50 of those windows are determined to correspond to a Sudanese ethnicity, the label(s) for the simulated dataset will reflect 25% English, 25% Japanese, and 50% Sudanese assignment.

The computing server 130 may adjust the weight parameters of the machine learning model based on the label comparisons between the predicted inheritance labels for the training samples and the actual ground-truth inheritance labels associated with the training samples. In some embodiments, the computing server 130 may backpropagate the errors to the machine learning model to adjust the parameters through machine learning techniques such as gradient descent. Alternatively, or additionally, the computing server 130 may adjust the weight parameters of the machine learning model based on the label comparisons by: comparing the predicted inheritance labels to the actual labels to identifying under-represented origins and over-represented origins; for an under-represented origin, increasing a value of the weight parameter corresponding to the under-represented origin; and for an over-represented origin, decreasing a value of the weight parameter corresponding to the over-represented origin.

In instances where certain origins that are associated with higher-than-average errors, the computing server 130 may identify those origins as under-represented or over-represented origins. An under-represented origin may correspond to scenarios where the machine learning model downplays or overlooks the impact of this origin in the genetic data, leading to predictions that miss these labels. An over-represented origin may indicate scenarios where the machine learning model over emphasizes this origin, possibly leading to predictions that assign these labels too frequently or inappropriately.

Upon identifying these under-represented and over-represented origins, the computing server 130 may adjust the values of their corresponding weight parameters. For an under-represented origin, the value of the corresponding weight parameter may be increased. This adjustment aims to make the model more sensitive to genetic data from this origin to improve correct predictions in subsequent runs. For an over-represented origin, the value of the associated weight parameter may be decreased to minimize the machine learning model's sensitivity towards this origin.

The computing server 130 may additionally tune a population-specific vector using, e.g., simulated inheritance datasets, for application with the machine learning model (step 324). The simulated inheritance datasets and associated ground-truth labels may be compared against labels predicted for the simulated inheritance datasets by the machine learning model, for example labels predicted by the machine learning model after adjusting the weight parameters of the machine learning model using label comparisons based on reference panel samples (step 322). The population-specific vector may be adjusted based on overprediction, underprediction, or accurate prediction of inheritance labels for simulated inheritance datasets that represent a true population composition of admixed individuals. For example, a population with high recall and low precision can be adjusted lower; a population with very high overlap scores (e.g. 5×) can be tuned lower; and/or a population with very low overlap scores (e.g. 20%) may be tuned higher. As a result of tuning per-population or origin-specific weight parameters based on label comparison for the simulated datasets, a tuned weight per population may be passed as a vector to an ethnicity estimation modality, e.g. the ethnicity estimation engine 245, for application with or against one or more of the nodes of the machine learning model. The use of the tuned weights per population advantageously improves the performance of the machine learning model while substantially reducing training and inference costs and time.

While the use of simulated inheritance datasets to tune the origin-specific weight parameters has been described, it will be appreciated that the disclosure is not limited thereto; rather, reference panel samples may be used instead of or in addition to the simulated inheritance datasets for tuning the population- or origin-specific weight parameters as suitable.

Using the trained machine learning model, including in embodiments the tuned origin-specific weight parameters, the machine learning model may be used to infer or determine inheritance labels for a new inheritance dataset (step 326). The machine learning model may receive, as an input, the new inheritance dataset, e.g., received from a new user of a genetic research service, and utilize the trained parameters for the model, including the origin-specific weight parameters, to predict an inheritance label for the inheritance dataset. In embodiments, the machine learning model may divide the received inheritance dataset into a plurality of windows and assign an inheritance label to each window. The predictions from the machine learning model may be output, e.g. displayed, to a user, for example by causing, using the computing server 130, a user device to display the predictions. Additionally, or alternatively, the computing server 130 may cause the predictions to be stored in, e.g., the genealogy data store 200, the genetic data store 205, and/or the individual profile store 210.

### Example Process for Selecting a Reference-Panel Individual

FIG. 4A is a flowchart depicting an example process 400 for selecting a reference-panel individual. The process 400 may be performed by one or more engines of the computing server 130 illustrated in FIG. 2, such as the ethnicity estimation engine 245. The process 400 may be embodied as a software algorithm that may be stored as computer instructions that are executable by one or more processors. The instructions, when executed by the processors, cause the processors to perform various steps in the process 400. In various embodiments, the process may include additional, fewer, or different steps. While various steps in process 400 may be performed with the use of computing server 130, each step may be performed by a different computing device. The process 400 may be an example of a process described in step 310 in FIG. 3. The process 400 may be particularly suited to generate a reference panel for an admixed origin, but the process 400 may also be used for generating non-admixed reference panels.

Reference-panel generation is a necessary process for many genetic analyses, including population genomics, but has historically been carried out manually and results in relatively few reference-panel samples in a reference panel, given the cost and labor associated with procuring and validating the samples. Even at the great cost and labor associated with curating the samples, error can still be introduced into downstream genetic analyses due to errors associated therewith, as self-reported family histories (which are used to determine that a person is representative of a single-origin member of a community or ethnicity corresponding to the reference panel) are error-prone. Further, the manually and somewhat arbitrarily drawn boundaries between communities and ethnicities can introduce error, as persons believed on the basis of such boundaries may be erroneously included in a reference panel. As such, there is a need for improved approaches to curating unbiased reference panels for downstream genetic analyses.

It has been advantageously found that matches between inheritance datasets may be relied upon to generate unbiased genetically driven reference panels, allowing for degrees of shared genetics to curate truly single-origin people in greater numbers and at lower cost and complexity than is possible using existing reference-panel curation approaches.

Inheritance labels (which may also be referred to as genetic labels) may be markers or identifiers in or based on a genetic dataset of an individual that denote specific attributes or characteristics of the individual. These labels may carry information about the geographic origin, ethnicity, or certain identifiable genetic traits tied to the individual. These labels may be used in different genetic analyses to identify ancestral connections, trace migration patterns, or even diagnose genetic disorders, among other applications.

In some embodiments, the computing server 130 accesses inheritance datasets from a database (step 410). The database may be the genetic data store 205 that includes a large number (e.g., millions or tens of millions) of inheritance datasets. Each inheritance dataset in the database may include one or more unique identifiers, which facilitates easier retrieval. The inheritance datasets may be organized based on specific categories such as geographical region, ethnicity, and genetic pool. After the inheritance datasets are successfully accessed and/or retrieved, the computing server 130 may process the inheritance datasets to prepare them for further analysis.

In an embodiment, inheritance labels regarding, e.g., communities, ethnicities, or other origins are mapped (step 412) to corresponding ethnic regions. For instance, one or more genetic communities—representing more-recent admixture and population events than ethnicity estimates—may be mapped to a pertinent, corresponding region or origin for determination of ethnicity. For purposes of discussion regarding the method 400, region and origin may be used interchangeably. For instance, a plurality of communities in the south of Italy and extending into Sicily may be consolidated in such a mapping to a Southern Italy ethnicity region for generation of a Southern Italy reference panel.

By facilitating such a mapping, the datasets associated with inheritance labels corresponding to the plurality of communities in the south of Italy and Sicily may be identified as potential candidates for the Southern Italy reference panel. This may be performed for any suitable grouping of individuals with already determined inheritance labels to a desired inheritance region, such as an ethnic region, and may be performed iteratively, such that as additional datasets are assigned to, e.g., the Southern Italy ethnicity region using the approaches described herein or to communities consolidated thereunder via the mapping described above, said datasets may themselves become potential candidates for, e.g., the South Italy reference panel. As such the reference panel(s) generated according to the process 400 may continually improve.

In some embodiments, the computing server 130 filters a plurality of candidates from the inheritance datasets (step 420). The computing server 130 may scan the retrieved datasets to identify specific inheritance labels. The inheritance labels may be distinctive to each dataset, or may be assigned to each dataset based on and from a predetermined set of inheritance labels. The inheritance labels may correspond to unique genetic characteristics or markers that are significantly associated with a particular origin. The markers may range from geographic location and ethnicity to specific genetic traits.

When filtering candidates based on inheritance labels, the computing server 130 may access a diverse pool of inheritance datasets. Each dataset may include one or more specific inheritance labels that denote origins, ethnicities, or specific genetic traits. The computing server 130 may set up specific filters according to the purpose of the filtering process. For example, if the purpose is to identify candidates from an ethnicity subregion, the filter may be an ethnicity filter that is set to retrieve inheritance labels that correspond to that specific ethnicity subregion. If the purpose is to identify candidates from a specific community, the filter may be a community filter that is set to retrieve inheritance labels that correspond to that specific community. Upon setting the filter, the computing server 130 may scan through each candidate to examine its associated inheritance labels. When the inheritance labels of a candidate match with the criteria set by the filter, that candidate is selected for further processing. Any candidate whose inheritance labels fail to match the criteria set by the filter is not selected.

In some embodiments, one or more filtering criteria may include a community filter, which may take the form of a normalized score (e.g., normalized to between 0 and 100) on how strongly the inheritance dataset of an individual is associated with a particular community. For example, if the normalized score is smaller than a predetermined threshold (e.g., 80), the data instance or candidate is filtered out. In embodiments, if the normalized score is equal to the predetermined threshold and the candidate is also associated with another community assignment, the candidate is filtered out. In embodiments, if the normalized score is equal to a higher predetermined threshold (e.g., 95) and the candidate is associated with another community assignment with a normalized score greater than a threshold (e.g. 20), the candidate is filtered out. It will be appreciated that these filtering scenarios are merely exemplary.

In some embodiments, one or more filtering criteria may also include ethnicity filter which may be applied previous to, instead of, in parallel with, or subsequent to the community signal filter. The computing server 130 may consider the samples in each community grouping and count the number of times each ethnicity occurs. The computing server 130 may construct allowed ethnicity thresholds for different ethnicities for each community. For example, in order to be qualified as a reference panel in a European community, the threshold amount of an Asian ethnicity contained in the inheritance dataset of an individual may be limited to a threshold. The threshold may be set manually or determined based on empirical data such as historical population composition data. Individuals, even for those who have high community signals to a particular community, may be filtered out if the individuals are associated with certain ethnicities that are not supposed to belong to the particular community. In embodiments, the ethnicity filter is configured to filter out candidates affiliated with two ethnicities that are greater than a threshold distance from each other, such as English and Japanese, as opposed to two ethnicities that are less than the threshold distance from each other, such as English and Swedish/Danish.

In some embodiments, one or more filtering criteria may also include a match filter, which may be applied previous to, instead of, in parallel with, or subsequent to the community signal filter and/or the ethnicity filter. FIG. 4C illustrates an example of a match filter, in accordance with some embodiments. The match filter may be used to identify suitable candidates from specific communities. For each candidate, the match filter calculates the IBD matches greater than a threshold (e.g., 10 cM) between the candidate and datasets associated with a given community. The match filter may additionally calculate the IBD matches to another set of reference panel individuals representing different communities or reference panels (i.e. different from the given community). The match filter may determine matches to another subset of reference panels or all or substantially all other reference panels.

After the IBD matches (or region matches) are determined, the match filter determines the proportion of the IBD matches that are included in the region (proportion region matches) for the corresponding community vs. matches outside the region. The graph 472 shows the in-region matches along the horizontal or X axis and the proportion of in-region matches to out-of-region matches along the vertical or Y axis. Out-of-region matches may be matches to a predefined subset of other reference panels or to all or substantially all other reference panels To further refine the selected candidates, a percentile cutoff 474, which may range from 10, 20, 30 . . . 90, etc., is set for each community or region. The graph 476 shows the percentile cutoff 474 applied to the graph 472. All candidates that fall below the cutoff, in terms of either the number of matches or the proportion of community-specific matches, may be removed from consideration. The data shows if individuals have a large number of IBD matches who are from an origin the individuals are likely to be the correct candidate to be selected in the reference panel corresponding to the origin as the number of matches a candidate has to a community is proportional to the amount of ethnicity that the candidate shares with the community.

Upon setting the filter, the computing server 130 may scan through each data instance in the genetic data store 205 to identify candidates who pass the filter(s), such as the community, ethnicity, and match filters. When the inheritance labels of a candidate match with the criteria set by the filter(s), that candidate is selected for further processing. Any candidate whose inheritance labels fail to match the criteria set by the filter(s) is not selected.

Referring back to FIG. 4A, in some embodiments, the computing server 130 identifies, for each filtered candidate, a number of the individuals in the particular origin whose inheritance datasets match the inheritance dataset of the candidate. For example, the computing server 130 may access each filtered candidate's inheritance dataset as well as the inheritance datasets of individuals from the specific origin. The computing server 130 may compare these datasets to determine matches between the candidate and the identified individuals. These matches may be based on various parameters such as IBD segments or other genetic markers specific to the origin. Each match may represent a degree of genetic alignment between the candidate dataset and the individuals from the specified origin.

For example, if Candidate A's inheritance dataset matches (e.g., via IBD match) with 50 individuals of a specific origin, the count for Candidate A is 50. An assessment may be run for all candidates in the candidate pool to identify how many matches each candidate has with the individuals of specific origins.

Referring to FIG. 4B, there is shown an example graph showing a genetic match correlation between individuals. Using Irish ethnicity as an example, it is observed that the percentage of Irish ethnicity in an individual's ethnicity estimate strongly correlates with the number of their genetic matches to other Irish individuals (considered here as “fourth cousins” or “fifth cousins”). For example, people who have 100% Irish ethnicity in their ethnicity estimate have many more matches compared to those with 50% or 25% Irish ethnicity. Thus, if an individual has many matches within a particular genetic community, this may suggest or confirm that they have a high proportion of their ethnicity estimate from that population. For example, this pattern may not only be found in Irish communities but also in other ethnicities like Italians and Jewish communities.

Candidates with the highest numbers of matches in the origin may be further selected as a further list of candidates who may be suited for inclusion in the reference panel corresponding to the origin.

In some embodiments, further filtering may be applied before the final candidates are selected to be included in the reference panel. For example, in some cases, a candidate's inheritance dataset may align with more than one origin. Referring back to FIG. 4A, in some embodiments, the computing server 130 determines that one of the candidates has a first number of matches associated with a particular origin and a second number of matches associated with a second origin (step 440). The computing server 130 may determine the number of genetic matches, such as IBD matches, that each candidate has within the various origins under consideration. For a given candidate, a first number of matches may be associated with a first origin (e.g., the particular origin), while a second number of matches may be associated with a second origin. The number of matches may correspond to the extent of genetic alignment between the candidate's dataset and the dataset associated with the various origins under consideration. If the second number of matches exceeds a predetermined threshold, the computing server 130 may determine that the candidate has significant genetic ties with the second origin. Based on this, the computing server 130 may remove the candidate from the initial candidate pool.

In this approach, the computing server 130 may determine the number of matches associated with each origin. For instance, a candidate may share a first number of matches with individuals from Origin A and a second number of matches with individuals from Origin B. By comparing the second number to a threshold, the computing server 130 may assess the extent to which of the candidate's genetics correspond to that second origin.

In some embodiments, in response to the prior step, the computing server 130 removes the candidates from the candidate pool (step 450). If the second number of matches exceeds a predetermined threshold, the computing server 130 may determine that the candidate has significant genetic ties with the second origin. In response, the computing server 130 may remove the candidate from the initial candidate pool. This approach may provide a way to identify candidates that have genetic ties with more than one origin. Multiple genetic ties may imply that the candidate has mixed ethnicity and/or that genetic variations common in one origin are shared with another. Filtering such individuals may reduce noise in the determination.

For example, the computing server 130 may generate a candidate pool for Northern Sweden (the first origin) that includes John, Sally, and Tom. The computing server 130 may determine that John has 12 (the first number) genetic matches with the dataset corresponding with Northern Sweden and 15 (the second number) matches with the dataset corresponding with Latvia (the second origin). If the preset threshold is 14, the computing server 130 may determine that the second number of matches for John exceeds that threshold. To maintain the accuracy of the Northern Sweden candidate pool, the computing server 130 may remove John from the candidate pool since his inheritance dataset appears to be potentially more indicative of the Latvian origin. In some embodiments, the computing server 130 may select a reference-panel individual according to the example process 400 illustrated in FIG. 4A of the present disclosure.

In some embodiments, the computing server 130 selects a candidate as a reference-panel individual based on the number of matched individuals that correspond to the candidate compared to numbers of matched individuals of other candidates (step 460). To select a candidate for the reference panel, the computing server 130 may compare the count of matches for each candidate. For example, a higher count may correspond to a higher degree of genetic alignment with the particular origin. If Candidate A has 100 matches and Candidate B has only 70, the computing server 130 may select Candidate A because it has more matches than Candidate B.

The computing server 130 may select a candidate as one of the reference-panel individuals based on the number of matched individuals for that candidate compared to numbers of matched individuals of other candidates. For example, the computing server 130 generates a reference panel for an origin such as Northern Sweden. The computing server 130 begins with a filtered pool of candidates such as Candidate 1, Candidate 2, Candidate 3 and Candidate 4. The computing server 130 identifies that the inheritance dataset of Candidate 1 matches with the inheritance datasets of 50 individuals for Northern Sweden. In the same way, the computing server 130 finds 70 matched individuals for Candidate 2, 65 matched individuals for Candidate 3 and 55 matched individuals for Candidate 4. Considering these numbers, Candidate 2 matches to the most individuals within the specified origin of Northern Sweden. In this scenario, the computing server 130 may select Candidate 2 as the top choice to be included as a reference-panel individual for the specified origin. This process may be repeated to select additional individuals for the reference panel to provide a thorough representation of the genetic variations within the specified origin, for example until a predefined number of reference-panel individuals have been selected.

FIG. 4A is a flowchart depicting an example process 400 for selecting a reference-panel individual. The process 400 may be performed by one or more engines of the computing server 130 illustrated in FIG. 2, such as the ethnicity estimation engine 245. The process 400 may be embodied as a software algorithm that may be stored as computer instructions that are executable by one or more processors. The instructions, when executed by the processors, cause the processors to perform various steps in the process 400. In various embodiments, the process may include additional, fewer, or different steps. While various steps in process 400 may be performed with the use of computing server 130, each step may be performed by a different computing device. The process 400 may be an example of a process described in step 310 in FIG. 3. The process 400 may be particularly suited to generate a reference panel for an admixed origin, but the process 400 may also be used for generating non-admixed reference panels.

Reference-panel generation is a necessary process for many genetic analyses, including population genomics, but has historically been carried out manually and results in relatively few reference-panel samples in a reference panel, given the cost and labor associated with procuring and validating the samples. Even at the great cost and labor associated with curating the samples, error can still be introduced into downstream genetic analyses due to errors associated therewith, as self-reported family histories (which are used to determine that a person is representative of a single-origin member of a community or ethnicity corresponding to the reference panel) are error-prone. Further, the manually and somewhat arbitrarily drawn boundaries between communities and ethnicities can introduce error, as persons believed on the basis of such boundaries may be erroneously included in a reference panel. As such, there is a need for improved approaches to curating unbiased reference panels for downstream genetic analyses.

It has been surprisingly found that matches between inheritance datasets advantageously may be relied upon to generate unbiased genetically driven reference panels, allowing for degrees of shared genetics to curate truly single-origin people in greater numbers and at lower cost and complexity than is possible using existing reference-panel curation approaches.

Inheritance labels (which may also be referred to as genetic labels) may be markers or identifiers in or based on a genetic dataset of an individual that denote specific attributes or characteristics of the individual. These labels may carry information about the geographic origin, ethnicity, or certain identifiable genetic traits tied to the individual. These labels may be used in different genetic analyses to identify ancestral connections, trace migration patterns, or even diagnose genetic disorders, among other applications.

In some embodiments, the computing server 130 accesses or receives inheritance datasets from a database. The database may be the genetic data store 205 that includes a large number (e.g., millions or tens of millions) of inheritance datasets. Each inheritance dataset in the database may include one or more unique identifiers, which facilitates easier retrieval. The inheritance datasets may be organized based on specific categories such as geographical region, ethnicity, and genetic pool. After the inheritance datasets are successfully retrieved, the computing server 130 may process the inheritance datasets to prepare them for further analysis. The inheritance datasets that are accessed may be datasets that are determined to be likely to correspond to single-origin individuals, e.g. inheritance datasets that are associated with a particular inheritance label, such as a geographical region or community, as determined by, e.g., the community assignment engine 230.

In an embodiment, inheritance labels regarding, e.g., communities, ethnicities, or other origins are mapped to corresponding ethnic regions (step 410). For instance, one or more genetic communities—representing more-recent admixture and population events than ethnicity estimates—may be mapped to a pertinent, corresponding region for determination of ethnicity. For instance, a plurality of communities in the south of Italy and extending into Sicily may be consolidated in such a mapping to a Southern Italy ethnicity region for generation of a Southern Italy reference panel.

By facilitating such a mapping, the datasets associated with inheritance labels corresponding to the plurality of communities in the south of Italy and Sicily may be identified as potential candidates for the Southern Italy reference panel, as such datasets may be considered based on their inheritance label and the corresponding mapping to be likely to be associated with single-origin individuals with deep roots to the Southern Italy ethnicity region. This may be performed for any suitable grouping of individuals with already determined inheritance labels to a desired inheritance region, such as an ethnic region, and may be performed iteratively, such that as additional datasets are assigned to, e.g., the Southern Italy ethnicity region using the approaches described herein or to communities consolidated thereunder via the mapping described above, said datasets may themselves become potential candidates for, e.g., the Southern Italy reference panel. As such the reference panel(s) generated according to the process 400 may continually improve.

In some embodiments, the computing server 130 filters a plurality of reference-panel candidates from the inheritance datasets (step 420). The computing server 130 may scan the retrieved datasets to identify specific inheritance labels. The inheritance labels may be distinctive to each dataset, or may be assigned to each dataset based on and from a predetermined set of inheritance labels, e.g. by the community assignment engine 230. The inheritance labels may correspond to unique genetic characteristics or markers that are significantly associated with a particular region. The markers may range from geographic location and ethnicity to specific genetic traits. In embodiments, the inheritance labels are assigned using an unsupervised process such as the Louvain method (described above) based on matches, e.g. IBD matches, detected between inheritance datasets. An inheritance dataset may be associated with a plurality of labels, including a community-specific label, an ethnicity-specific label, or others as suitable.

When filtering candidates based on inheritance labels, the computing server 130 may access a diverse pool of inheritance datasets. Each dataset may include one or more specific inheritance labels that denote origins, ethnicities, or specific genetic traits. The computing server 130 may set up specific filters according to the purpose of the filtering process. For example, if the purpose is to identify candidates from an ethnicity subregion, the filter may be an ethnicity filter that is set to retrieve inheritance labels that correspond to that specific ethnicity subregion. If the purpose is to identify candidates from a specific community, the filter may be a community filter that is set to retrieve inheritance labels that correspond to that specific community. Upon setting the filter, the computing server 130 may scan through each candidate to examine its associated inheritance labels. When the inheritance labels of a candidate match with the criteria set by the filter, that candidate is selected for further processing. Any candidate whose inheritance labels fail to match the criteria set by the filter is not selected.

Referring back to FIG. 4A, in some embodiments, the computing server 130 applies, for each identified reference-panel candidate, a community-assignment filter (step 422). The community-assignment filter may take the form of a normalized score (e.g., normalized to between 0 and 100) on how strongly the inheritance dataset of an individual is associated with a particular community. The score may be determined using the community assignment engine 230. For example, if the normalized score is smaller than a predetermined threshold (e.g., 80), the data instance or candidate is filtered out. In embodiments, if the normalized score is equal to the predetermined threshold and the candidate is also associated with another community assignment, the candidate is filtered out. In embodiments, if the normalized score is equal to a higher predetermined threshold (e.g. 95) and the candidate is associated with another community assignment with a normalized score greater than a threshold (e.g. 20), the candidate is filtered out. It will be appreciated that these filtering scenarios are merely exemplary and that any community-specific filter may be applied.

In some embodiments, the computing server 130 applies, for each identified reference-panel candidate, an ethnicity-assignment filter (step 424). The ethnicity-assignment filter may be applied previous to, instead of, in parallel with, or subsequent to the community signal filter. The computing server 130 may consider the samples in each community grouping and count the number of times each ethnicity occurs, as determined by, e.g., the ethnicity estimate engine 245. The computing server 130 may construct allowed ethnicity thresholds for different ethnicities for each community or region. For example, in order to be qualified as a reference-panel individual in a European community, the threshold amount of an Asian ethnicity contained in the inheritance dataset of an individual may be limited to a threshold. The threshold may be set manually or determined based on empirical data such as historical population-composition data. Individuals, even for those who have high community signals to a particular region, may be filtered out if the individuals are associated with certain ethnicities that are not considered to belong to the particular community (and therefore indicate an unacceptably high degree of admixture for a single-origin person). In embodiments, the ethnicity filter is configured to filter out candidates affiliated with two ethnicities that are greater than a threshold geographic distance from each other, such as English and Japanese, as opposed to two ethnicities that are less than the threshold geographic distance from each other, such as English and Swedish/Danish.

In some embodiments, the computing server 130 applies, for each identified reference-panel candidate, a matches filter (step 440). The matches filter may be applied previous to, instead of, in parallel with, or subsequent to the community signal filter and/or the ethnicity filter. FIG. 4C illustrates an example of a matches filter, in accordance with some embodiments. The matches filter may be used to identify suitable candidates from specific communities or regions. For each candidate, the matches filter calculates the IBD matches greater than a threshold (e.g., 10 cM) between the candidate and datasets associated with a given community or region. After the IBD matches (or region matches) are determined, the matches filter determines the proportion of these IBD matches that are included in the region (proportion region matches) for the corresponding community or region vs. matches outside the region. The graph 472 shows the in-region matches along the horizontal or X axis and the proportion of in-region matches to out-of-region matches along the vertical or Y axis. To further refine the selected candidates, a percentile cutoff 474, which may range from 10, 20, 30 . . . 90, etc., is set for each community or region. The graph 476 shows the percentile cutoff 474 applied to the graph 472. All candidates that fall below the cutoff, in terms of either the number of matches or the proportion of community-specific matches, may be removed from consideration. The data show that if individuals have a large number of IBD matches who are from a region the individuals are likely to be the correct candidate to be selected in the reference panel corresponding to the region as the number of matches a candidate has to a community or region is proportional to the amount of ethnicity that the candidate shares with the community or region. This approach advantageously utilizes, while addressing a plurality of regions simultaneously, a proportion of in-region vs. out-of-region matches such that the proportion that most closely aligns with a user's probable ethnicity is accounted for and utilized to improve accuracy.

Alternatively, or additionally, the matches filter may utilize a comparison of a number of matches between a candidate and datasets associated with one or more regions. Referring to FIG. 4B, there is shown an example graph showing a genetic match correlation between individuals. Using Irish ethnicity as an example, it is observed that the percentage of Irish ethnicity in an individual's ethnicity estimate strongly correlates with the number of their genetic matches to other Irish individuals (considered here as “fourth cousins” or “fifth cousins”). For example, people who have 100% Irish ethnicity in their ethnicity estimate have many more matches compared to those with 50% or 25% Irish ethnicity. Thus, if an individual has many matches within a particular genetic community, this may suggest or confirm that they have a high proportion of their ethnicity estimate from that population. For example, this pattern may not only be found in Irish communities but also in other ethnicities like Italians and Jewish communities.

Candidates with the highest numbers of matches in the region may be further selected as a further list of candidates who may be suited for inclusion in the reference panel corresponding to the region.

In some embodiments, further filtering may be applied before the final candidates are selected to be included in the reference panel. For example, in some cases, a candidate's inheritance dataset may align with more than one region. Referring back to FIG. 4A, in some embodiments, the computing server 130 determines that one of the candidates has a first number of matches associated with a particular region and a second number of matches associated with a second region (step 440). The computing server 130 may determine the number of genetic matches, such as IBD matches, that each candidate has within the various regions under consideration. For a given candidate, a first number of matches may be associated with a first region (e.g., the particular region), while a second number of matches may be associated with a second region. The number of matches may correspond to the extent of genetic alignment between the candidate's dataset and the dataset associated with the various regions under consideration. If the second number of matches exceeds a predetermined threshold, the computing server 130 may determine that the candidate has significant genetic ties with the second region. Based on this, the computing server 130 may remove the candidate from the initial candidate pool.

In this approach, the computing server 130 may determine the number of matches associated with each region. For instance, a candidate may share a first number of matches with individuals from Region A and a second number of matches with individuals from Region B. By comparing the second number to a threshold, the computing server 130 may assess the extent to which of the candidate's genetics correspond to that second region.

In some embodiments, in response to the prior step, the computing server 130 removes the candidates from the candidate pool (step 450). If the second number of matches exceeds a predetermined threshold, the computing server 130 may determine that the candidate has significant genetic ties with the second region. In response, the computing server 130 may remove the candidate from the initial candidate pool. This approach may provide a way to identify candidates that have genetic ties with more than one region. Multiple genetic ties may imply that the candidate has mixed ethnicity and/or that genetic variations common in one region are shared with another. Filtering such individuals may reduce noise in the determination.

For example, the computing server 130 may generate a candidate pool for Northern Sweden (the first region) that includes individuals named John, Sally, and Tom. The computing server 130 may determine that John has 12 (the first number) genetic matches with the dataset corresponding with Northern Sweden and 15 (the second number) matches with the dataset corresponding with Latvia (the second region). If the preset threshold is 14, the computing server 130 may determine that the second number of matches for John exceeds that threshold. To maintain the accuracy of the Northern Sweden candidate pool, the computing server 130 may remove John from the candidate pool since his inheritance dataset appears to be potentially more indicative of the Latvian region. In some embodiments, the computing server 130 may select a reference-panel individual according to the example process 400 illustrated in FIG. 4A of the present disclosure.

In some embodiments, the computing server 130 selects a candidate as a reference-panel individual based on the number of matched individuals that correspond to the candidate compared to numbers of matched individuals of other candidates. To select a candidate for the reference panel, the computing server 130 may compare the count of matches for each candidate. For example, a higher count may correspond to a higher degree of genetic alignment with the particular region. If Candidate A has 100 matches and Candidate B has only 70, the computing server 130 may select Candidate A because it has more matches than Candidate B.

The computing server 130 may select a candidate as one of the reference-panel individuals based on the number of matched individuals for that candidate compared to numbers of matched individuals of other candidates. For example, the computing server 130 generates a reference panel for a region such as Northern Sweden. The computing server 130 begins with a filtered pool of candidates such as Candidate 1, Candidate 2, Candidate 3 and Candidate 4. The computing server 130 identifies that the inheritance dataset of Candidate 1 matches with the inheritance datasets of 50 individuals for Northern Sweden. In the same way, the computing server 130 finds 70 matched individuals for Candidate 2, 65 matched individuals for Candidate 3 and 55 matched individuals for Candidate 4. Considering these numbers, Candidate 2 matches to the most individuals within the specified region of Northern Sweden. In this scenario, the computing server 130 may select Candidate 2 as the top choice to be included as a reference-panel individual for the specified region. This process may be repeated to select additional individuals for the reference panel to provide a thorough representation of the genetic variations within the specified region, for example until a predefined number of reference-panel individuals have been selected.

Upon setting one or more of the above-mentioned filters, the computing server 130 may scan through each data instance in the genetic data store 205 to identify candidates who pass the filter(s), such as the community, ethnicity, and match filters. When the inheritance labels of a candidate fail to match with the criteria set by the filter(s), that candidate is removed from the candidate pool (step 450).

Filtered reference-panel candidates may be further assessed by plotting birth locations of the filtered reference-panel candidates (step 460). The birth locations may be ascertained from pedigrees associated with the reference-panel candidates. The pedigrees may be accessed, in embodiments, from the genealogy data store 200. Birth locations associated with the pedigrees may be determined based on, e.g., a birth-related record and/or birth-specific data in a pertinent node of the pedigree. Where, for example, a pertinent node of a pedigree is updated to state that a birth location for an inheritance dataset is Cambridge, England, a geographic identifier associated with and specific to Cambridge, England is accessed by the computing server 130 and plotted. The pertinent node may be the node of a pedigree specific to the dataset, e.g. a person of interest. The computing server 130 may access geographic identifiers from pertinent nodes for each pedigree for filtered reference-panel candidates and plot the geographic identifiers to identify outliers. Where one or more plotted geographic identifiers varies from, e.g., a centroid of the plotted geographic identifiers by more than a threshold amount or proportion, the corresponding reference-panel candidates may be removed. This advantageously reduces noise in the downstream assignment of datasets to the region for the reference-panel by ensuring that non-single-origin persons and/or non-members of the region are not erroneously included in the comparison. While birth location has been described, it will be appreciated that the disclosure is not limited thereto and that any suitable datum or data from one, more, or all of the filtered candidates may be utilized as suitable to assess for accuracy.

After applying filters to the selected reference-panel candidates and assessing the filtered candidates by birth location, remaining reference-panel candidates may be selected as reference-panel individuals.

In embodiments, the construction of a reference panel for a region from the identified, filtered, and selected candidates may incorporate individuals from a plurality of distinct communities and/or ethnicities. For example, a Southern Italy ethnicity region may overlap with dozens of genetic communities, such as communities corresponding to Apulia, Calabria, Central Eastern Italy, Central Southwest Italy, Northern Sicily, and Southeastern Sicily. Assembling selected reference-panel candidates who correspond variously to such constituent regions may be performed according to several strategies. In one approach, the selected individuals are sampled randomly for inclusion in a reference panel without respect to their constituent-region assignment. It has been surprisingly found that in many regions this approach maximized performance.

In other embodiments, the reference panel is assembled by sampling selected candidates from constituent regions according to a proportion of representation or population of said constituent regions. For example, where constituent regions A, B, and C are included within a region X, and where the populations of constituent regions A and B account for 40% each of the population of region X, 40% of the reference panel may be drawn from selected candidates corresponding to constituent regions A and B and 20% of the reference panel may be drawn from selected candidates corresponding to constituent region C.

In other embodiments, the reference panel is assembled by selecting an equal number of samples from each of the constituent regions that are rolled up under a region. It has been surprisingly found that this approach improves performance in regions that are highly differentiated internally, with subethnic groups being highly distinct from each other, and/or in regions where subethnic groups have uneven sampling. In other embodiments, the reference panel is assembled by overselecting or oversampling candidates from a constituent region determined to be at a geographic center of the region, and undersampling from outlying constituent regions. In embodiments, a plurality of construction approaches are utilized in parallel, with a best-performing approach selected for each region.

The process of evaluating candidates according to embodiments of the present disclosure advantageously reduces the cost of curation and improves the accuracy of a reference-panel by utilizing genetic metrics using an orders-of-magnitude larger scale of datasets rather than manual assessments like modern geographic boundaries per previous approaches to determine representatives of a genetic origin.

Example Process for Generating Training Samples from a Plurality of Simulated Individuals

FIG. 5A is a flowchart depicting an example process 500 for generating training samples from a plurality of simulated individuals, in accordance with some embodiments. The generated training samples may be used for training and evaluating the performance of a machine learning model, such as the ethnicity estimation engine 245. It will be appreciated that while training samples are described, the disclosure is not limited to generating training samples for a machine learning model for predicting inheritance labels for inheritance datasets from simulated individuals, but rather the simulated individuals additionally or alternatively may be generated so as to train, in embodiments, population-specific weight vector(s) which may be applied to a pre-trained or separately trained machine learning model for predicting inheritance labels for inheritance datasets.

The process 500 may be performed by one or more engines of the computing server 130 illustrated in FIG. 2. The process 500 may be embodied as a software algorithm that may be stored as computer instructions that are executable by one or more processors. The instructions, when executed by the processors, cause the processors to perform various steps in the process 500. In various embodiments, the process may include additional, fewer, or different steps. While various steps in process 500 may be performed with the use of computing server 130, each step may be performed by a different computing device. The process 500 may be an example process that corresponds to step 312 and step 314 described in FIG. 3.

In embodiments, the process 500 facilitates the generation of reconstructed realistic pedigrees for the generation of simulated data for ethnicity or other genetic assignment predictions. The process 500 may utilize or generate population compositions that facilitate population-specific seeding of a simulated pedigree using reference-panel individuals (in embodiments, reference-panel individuals from reference panels constructed according to the above-mentioned approach) for generation of simulated datasets that comprise trustworthy and accurate labels for training a machine learning model.

In some embodiments, the computing server 130 may access a population composition of a geographical location (step 510). The population composition may correspond to the specific mix of genetic profiles from the geographical area. The population composition may be determined based on genetic data and/or genealogy data. By way of example, the computing server 130 may access the population composition of the geographical location via connection to a genetics database to access the required information. The database may include a broad range of genetic information about individuals from the geographical location. The information may include data on individuals' family history (genealogical lineage or descent), ethnicity (cultural factors such as their nationality or tribal affiliation), genetic variants (distinct versions of the same gene or differences in the DNA sequence within a population), and/or other relevant genetic data. In some embodiments, the population composition may also be determined from genealogy data such as census data from the genealogy data store 200.

In embodiments, the computing server 130 may determine a population composition. Determining a population composition may comprise utilizing family trees from a particular population. It has been found that while family trees individually are unreliable given the prevalence of human error, false paternity, family lore, adoption events, high number of generations in an admixed country like the United States before getting to ethnic roots in Europe or elsewhere, lack of records (e.g. due to trans-ocean migrations) and other challenges that make it difficult for individuals to accurately reflect their true family history through a number of generations, family trees in aggregate yield data that, when used in a simulation as described herein, assign accurate labels. That is, by aggregating family trees associated with a population, patterns may be deduced therefrom so as to accurately reflect a population composition and simulate inheritance datasets with accurate labels for better training a machine learning model to predict, e.g., ethnicity or other labels for downstream datasets.

For example, a plurality of trees from a one or more populations of interest may be accessed from a storage medium, such as the genetic data store 205. The populations of interest and constituents thereof may be determined using, e.g., inheritance labels such as an assignment to a community based on DNA. That is, for one or more communities of interest, trees may be obtained from a database based on the trees being associated with an individual who, on the basis of a DNA result or analysis, has been determined to be a member of one or more of the communities of interest. The plurality of trees may comprise information regarding names, dates, and locations, such as birth dates and locations, and may extend a plurality of generations vertically. The locations may be mapped to, e.g., countries. Countries may be, in turn, labeled or be associated with labels as “admixed” or “non-admixed.” For example, the United States may be labeled as an admixed country, while Sweden may be labeled as non-admixed.

The accessed trees may be read by the computing server 130 so as to identify a country where each ancestor at each generation, up to a predetermined number of generations, was born. For each of the accessed trees, a person of interest or bottommost generation (i.e. the tree owner or creator) may be the first generation, and the tree may be read up to, e.g., an eighth generation from the person of interest. The number of generations relative to the bottommost generation may be assessed vis-à-vis the identified countries, allowing the computing server 130 to determine a generation-specific population composition from the accessed trees. Alternatively, or additionally, a birth year may be assessed with the location and utilized to categorize tree persons from the one or more populations of interest.

The population composition, accordingly, may allow for generating a simulated data tree for a simulated person of interest for a location. For example, to generate training samples for a community such as Victoria, Australia, the computing server 130 may identify a plurality of members of communities mapped to the Victoria, Australia region. Trees associated with the identified plurality of members of the corresponding communities may be accessed and read by the computing server 130 to determine a population composition for the Victoria, Australia region.

The aggregated trees may be traversed by the computing server 130 to identify a proportion of ancestors born in particular locations at each generation relative to the persons of interest of the aggregated trees. For example, at a second generation of the aggregated trees (i.e. the parents of the persons of interest), the computing server 130 may determine based on the birth locations or other data in the pertinent nodes of the aggregated trees that a particular proportion of ancestors of the community may have been born at a variety of locations, and so on up through, e.g., an eighth generation. A population composition for a particular region generated according to the described modality is shown in FIG. 5B, where Prop1 represents the proportions of ancestors in the aggregated trees born at various regions at a first generation, Prop2 represents the proportions of ancestors in the aggregated trees born at various regions at a second generation, and so on. This may be utilized, in combination with the country labels (as admixed or non-admixed) to determine a generation- or years-specific probability that an ancestor is a founder, and a population distribution of founder ancestors.

In some embodiments, the computing server 130 may sample, based on the population composition of the geographical location, reference-panel individuals from the plurality of reference panels (step 520). For example, if the population composition data shows that a large percentage of the population in the geographical location has ethnicity from or roots to a particular ethnic group or region, the computing server 130 may sample more individuals from the reference panel that represents that ethnic group or region. If the population composition includes generation-specific compositions (for example, certain genetic markers or traits that have developed or become more prevalent in recent generations), then these factors may also guide the sampling process. The computing server 130 may sample more individuals who represent these newer, generation-specific compositions.

If some genetic profiles are dominant within certain specific geographical locations, the sampling may account for these characteristics to provide a variety of representation in the simulated data tree. The sampled individuals may be placed within the simulated data tree to provide a correlation of genetic markers with family relations and demographic indicators.

In some embodiments, the computing server 130 may represent sampled reference-panel individuals in nodes of a simulated data tree (step 530). The simulated data tree may be a graphical visualization or model that captures and organizes the genetic information of the sampled individuals. Each individual sampled from the reference panel may be represented by a specific node in the data tree. The placement of the individuals within the data tree may depend on factors such as generation-specific compositions. For instance, an individual from an older generation (e.g., a founder) may be placed at a higher position in the data tree, while another individual from a more recent generation may be represented at a lower level. This structured arrangement may provide for visual representation of relationships between different generations and information on how certain genetic markers have been passed down or changed over generations in that specific geographical location. Other factors may be considered such as specific genetic markers, the prevalence of certain traits within a geographical location, etc. when determining the placement of individuals within the nodes of the simulated data tree.

By way of example, each selected reference-panel individual in the simulated data tree may be represented by a node, and the connections (or branches or edges) between the nodes may indicate familial relationships such as parent-child or sibling relationships. Ancestor nodes may be at the higher or upstream levels (origin or root) of the simulated data tree. The ancestor nodes may represent the founders of families. In one example, the ancestor nodes may be drawn from reference panels. The nodes that are downstream of the ancestor nodes may be descendant nodes. The descendant nodes may represent the offspring of the founders, and subsequently their offspring, and so on. In one approach, a simulation may determine the status of each node as a founder or non-founder and assign it a genetic makeup based on a set of predefined probabilities and the genetic makeups of the preceding nodes (or ancestors).

The founder may be considered the originator of the genetic line in the data tree. The founder may be an individual from whom any significant fraction of the lineage's genes can be traced. For example, founders may be determined based on a probability pattern calculated from real family trees. For example, if an individual's parents are not founders, an algorithm recursively may move up or expand and populate the generations until a founder is identified.

For example, a generational probability of an ancestor being a founder and a population distribution of founder ancestors may be relied upon to determine whether a particular ancestor in the simulated data tree is a founder. At each node of the tree, starting from a person of interest or bottommost generation, the computing server 130 may determine whether a tree node, e.g., the parents of a pertinent node in the preceding generation, is a founder. If, based on the generational probability of an ancestor being a founder and on the population distribution of founder ancestors for the region, the parent is likely to have been born in a country labeled as non-admixed, the parent is labeled as a founder, and further recursion is not performed on that line or branch of the tree. If, based on the generational probability of an ancestor being a founder and on the population distribution of founder ancestors for the region, the parent is likely to have been born in a country labeled as admixed, the parent is not labeled as a founder, and further recursion is performed for that parent's parents to arrive at founder ancestors on the pertinent lines of the tree. Where the parent is labeled as a founder, the country of birth may be assigned based on the population composition at that particular generation. For example, using the population composition of FIG. 5B, a founder ancestor at generation three may have a 49% chance of being assigned as English by birth. This allows the computing server 130 to select a dataset of a reference-panel individual from the England reference panel to seed this node in an admixture simulation.

As discussed immediately above, the computing server 130 may sample the reference-panel individuals from the plurality of reference panels and represent them in the nodes of the simulated data tree. The computing server 130 may sample, based on the population composition of the geographical location, reference-panel individuals from the plurality of reference panels for the plurality of origins. In one approach, selection of the reference-panel individuals may be based on a variety of factors, such as generation-specific compositions. The computing server 130 may represent the sampled reference-panel individuals in the nodes of the particular simulated data tree by selecting placements of the sampled reference-panel individuals based on the generation-specific composition. For example, the genetic and demographic markers in a specific generation may differ from those in another due to various factors like genetic mutations, migratory patterns, intermarriage, etc. Each sample from a reference panel may represent an individual with certain genetic and demographic attributes belonging to a specific generation.

FIG. 5B shows the probabilities used to generate a simulated data tree. Each branch of the simulated data tree may represent a share of the simulated data tree, which could be based on geographic factors (or other demographic distinctions). A proportion is calculated and/or determined at or for every generation. The determination of the proportions is made by collating where the ancestors were born at each stage of the generational lineage, then these places of birth are categorized into distinct regions (e.g., Cornwall, England, France, etc.). The distribution of these regions at each generation level is the trees proportion (e.g., Prop1 for the proportion or distribution at generation one (e.g., a topmost generation), Prop2 for the proportion or distribution at generation two, Prop3 for the proportion or distribution at generation three, etc.).

That is, a component of the ethnicity estimation engine 245 may be configured to utilize the probabilities in the table of FIG. 5B to select ancestors for a desired number of simulated data trees, e.g., 200 simulated data trees, where, in embodiments, the probabilities are used with a normal distribution and a probability to sample source populations, e.g. reference panels, for founders/ancestors. The data in FIG. 5B may be complemented by data indicating the probability of a founder in each generation of a simulated data tree. The use of the probabilities in FIB. 5B and the probabilities of a founder in each generation, which may be based on observable, real data, to generate probabilistic simulations to create a plurality of renditions of possible trees, advantageously allows for the simulation of admixed descendants of admixed populations, enabling the systems, methods, and computer-program product embodiments of the disclosure to facilitate the addition of new regions while improving accuracy of predictions generated for new inheritance datasets.

The computing server 130 may account for this generation-specific context while positioning individuals in the nodes of the data tree. For example, an individual from a reference panel may belong to the first generation and have a genetic marker A. The computing server 130 may place this individual at a position within the data tree representing the first generation. If a second-generation individual from the reference panel has genetic marker B, the computing server 130 may place this second-generation individual at a different level in the tree, indicating the second generation.

FIG. 5C shows a simulated data tree 572 generated from the proportions of FIG. 5B. This data tree provides a simulation of genetic variation and distribution seen in actual family trees. The table 574 provides genetic distribution data of ancestral categories of the simulated tree.

The purpose of constructing a simulated data tree is to create a probabilistic model that predicts an individual's genetic lineage and expected distribution of genes. For example, with respect to a particular origin, the simulated data tree may reflect what a real family tree for an individual from that particular origin look like. In one approach, actual family trees may be used to create probability vectors for the population composition, which represent the likelihood of an ancestor being a founder. The probability vector may be population-specific.

New probability vectors may be constructed for each generation. For example, if an individual is simulated to be a founder at generation one, there may be a distribution probability to determine which population they come from, for instance, if they are from England, France, etc. One approach includes simulating seven generations of admixed individuals to create simulated individuals for each population of the admixture. The admixture may refer to the process of combining of genetic traits from two or more populations to create a new hybrid population. Seven generations of admixture may refer to the blending of genetic traits that has occurred over seven generational transitions.

Simulating these generations may provide the creation of simulated individuals for each population involved in the admixture. For example, if a father is determined to be a founder of generation one in a data tree, there may be a probability that he comes from a plurality of countries. In building a simulated data tree, an individual from a reference panel of one of the countries may be chosen to represent the founder. The simulated data trees may mimic the real-world genetics of actual populations, helping researchers understand and/or predict their genetic compositions. After these simulated individuals are created, their genetic information may be tested against those of real individuals for accuracy.

Continuing to refer to FIG. 5A, in some embodiments, the computing server 130 may generate a simulated inheritance dataset representing a simulated individual in a simulated data tree (step 540). The computing server 130 may generate the simulated individual as a descendant of the selected and situated reference-panel individuals. The computing server 130 may generate the inheritance dataset of the simulated individual based on the reference-panel individuals on the data tree and inheritance events. An example of this process is provided in and described in regard to step 314 of FIG. 3. The generation of the simulated inheritance dataset may be performed according to and/or using any suitable modality, including suitable admixture simulation approaches. The generation of the simulated data tree according to the disclosure advantageously ensures that the simulated inheritance dataset is reflective of actual population patterns, which has been found to improve accuracy of ethnicity estimate generation for downstream datasets, e.g. real-user datasets.

In some embodiments, the computing server 130 may generate per-window origin labels of the simulated inheritance dataset (step 550). The computing server 130 may divide the inheritance dataset into smaller units, referred to as windows. Each window may provide a smaller portion of genetic data.

Within each window, the computing server 130 may determine how a segment of the inheritance dataset is inherited. The computing server 130 may identify the reference-panel individual in the data tree who passes down the genetic segment to the simulated inheritance dataset. The identified segment may correspond to a sequence of genetic information within the inheritance dataset which the reference-panel individual is the genetic source of. The computing server 130 may determines the origin label of the identified reference-panel individual such that the origin label corresponds to a geographical, ethnic, or ancestral group to which the individual belongs. The determined origin label may be used as the actual label for the inheritance dataset within that window. Thus, the actual label may represent the original ancestor from which the genetic segment in the window in question was inherited.

The computing server 130 may simulate a plurality of inheritance events. The inheritance events may include simulated meiosis and simulated recombination based on linkage disequilibrium. With respect to meiosis, it may refer to a type of cell division in sexually reproducing organisms resulting in gametes or sex cells (such as sperm or egg cells) with half the number of chromosomes. This halving may facilitate the fusion of male and female gametes result in offspring with the correct total number of chromosomes. The simulation of this process in the inheritance events may provide the model to account for the way genetic information passes from parents to offspring. The simulation may divide the genetic profile of an individual node or reference-panel individual by half, just like the transmission of genetic information during reproduction.

With respect to simulated recombination based on linkage disequilibrium, it is the process in which genetic material gets reshuffled during the formation of gametes. For example, through recombination, genes from both parents get mixed. The simulation may generate or simulate this reshuffling effect to provide more varied and realistic genetic profiles for the simulated individuals. Linkage disequilibrium may come into play when the system simulates recombination events. A linkage disequilibrium may refer to a scenario where certain genes located close together on a chromosome tend to be inherited together more frequently than would be expected if their assortment were entirely random. The simulation of recombination events may be constrained by or reflect this principle.

When simulating the inheritance events, the computing model may account for the reality that bits of genetic data are passed onto the simulated descendants not just randomly, but according to patterns observed in real-world genetics. That is, genes that are often inherited together in actual populations may be inherited together in the simulation as well. The simulation may use these patterns of linkage disequilibrium to provide which genes are passed onto descendants in the simulated data tree.

The computing server 130 may generate the particular simulated inheritance dataset of the particular simulated individual based on the plurality of inheritance events. The computing server 130 may generates the simulated inheritance dataset of the simulated individual based on the plurality of inheritance events. For example, if an individual is determined to be a descendant of a particular reference panel that represents a specific geographical location, the simulated inheritance events may provide the genetic traits associated with that location. This dynamic nature of the simulated inheritance dataset provides a realistic tool for understanding the genetic makeup of individuals in specific demographic groups or geographical regions.

In some embodiments, the computing server 130 may generate training samples from a plurality of simulated individuals (step 560). The process of generating the simulated inheritance datasets may include simulating a descendant of the reference-panel individuals who are placed in the particular simulated data tree to generate an inheritance dataset of the descendant (e.g., a simulated genome or a simulated genetic dataset of the descendant), simulating a plurality of inheritance events such as reproduction events based on how the family tree is simulated (e.g., a simulation of a reproduction event between a first reference-panel individual simulated as father and a second reference-panel individual simulated as mother), generating the particular simulated inheritance dataset of the particular simulated descendant based on the plurality of inheritance events, and repeating the process to generate additional descendants. The simulated descendants' inheritance datasets may be generated based on a number of reproduction events such as between great grandparents, grandparents, and parents. The reproduction events may involve simulation of meiosis and recombination events based on linkage disequilibrium. The generation of a plurality of simulated inheritance datasets may be similar to a Monte Carlo simulation. For example, for a particular simulated data tree, different inheritance datasets of different descendant may be simulated with the same combinations of reference-panel individuals as simulated relatives using different meiosis events and recombination events. Additionally, or alternatively, a number of different simulated data trees may be generated using the same population composition by randomly drawing reference-panel individuals based on the population composition. In turn, each simulated data tree may be used to generate one or more simulated descendants.

The computing server 130 may transform the inheritance datasets of the simulated individuals into training samples for training and evaluating the performance of a machine learning model. The transformation process may depend on the data format acceptable by the machine learning model. In one approach, the computing server 130 may convert the inheritance datasets into a matrix where each row corresponds to a simulated individual and each column represents a specific genetic characteristic. This format may provide each simulated individual's complete genetic profile to be represented as a single row within the matrix.

The inheritance datasets of the simulated datasets may be passed through a suitable modality such as the ethnicity estimation engine 245 and then compared against other results/datasets to determine overlap with an expected distribution.

It will be appreciated that while reconstructed realistic pedigrees are discussed herein in regard to generating improved training data for ethnicity assignments, the disclosure is not limited thereto but rather may be widely applied to, e.g., traits predictions, community assignments, automated pedigree generation, and other applications as suitable.

### Example of a Hidden Markov Model (HMM)

FIG. 6 illustrates a simplified example of an inter-window HMM 600, in accordance with some embodiments. The inter-window HMM 600 may be used in the ethnicity estimation engine 245 and used as the machine learning model discussed in process 300 and process 500 that is trained using the training samples generated by the process 500.

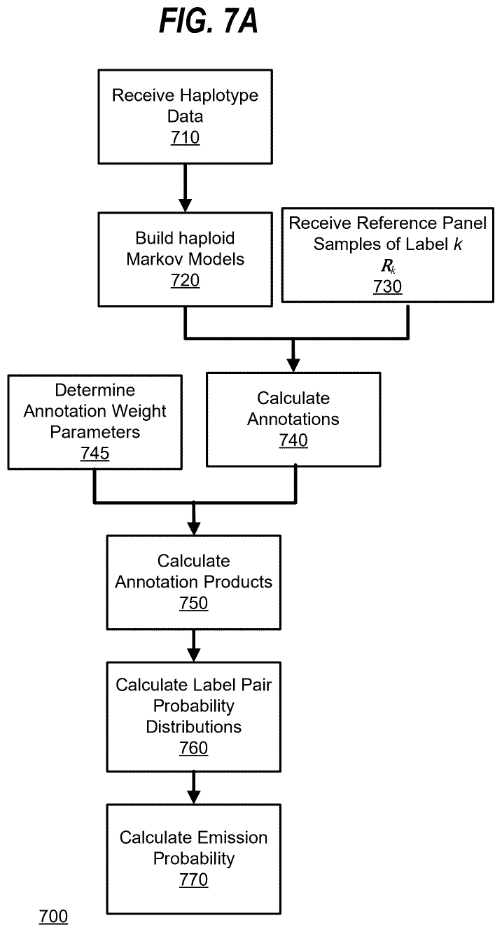

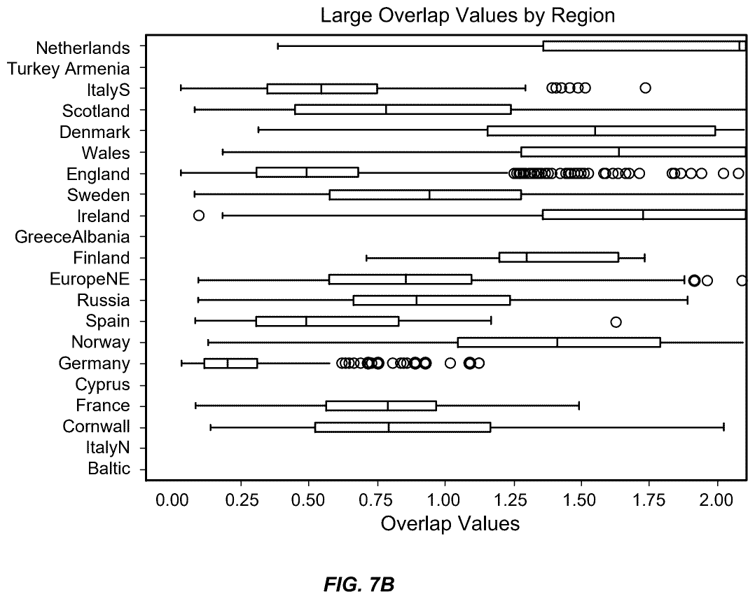

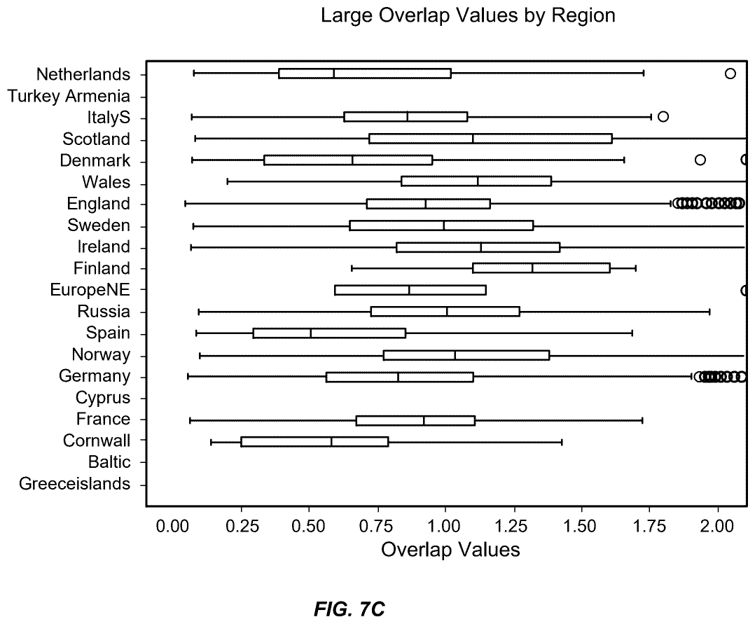

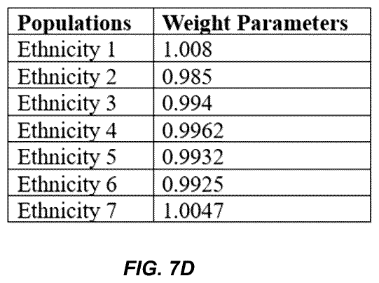

The inter-window HMM 600 may be a directed (e.g., in the direction from left to right as shown in FIG. 6) acyclic graph that includes a plurality of node groups. The graph representing the inter-window HMM 600 may also be referred to as a trellis. Graphically, each node group in the trellis may also be referred to as a level, a slot, a graph window, or a layer. Each node group represents a window w that corresponds to a genetic segment such as a set of SNPs. A plurality of nodes (represented by the circles in FIG. 6) are arranged in each node group. Each node represents a possible state of the window w. Each node is associated with an emission probability representing a likelihood of the window is observed as having a particular pair of phased haplotype datasets given the window is having the hidden state (i.e., the window is assigned with a particular pair of labels). In other words, the particular pair of phased haplotype datasets may be an observation in a hidden Markov model while the state that is labeled may be the “hidden” state of the hidden Markov model because the labels are not apparent given only the genotype dataset or the phased haplotype dataset. The inter-window HMM 600 also includes a plurality of edges. Each edge connects a first node of a first node group to a second node of a second node group. Each edge represents a transition from the first node of the first node group to the second node of the second node group. Each edge is associated with a transition probability that represents a likelihood of transition from the first node to the second node. The determination of the emission probabilities and transition probabilities will be discussed in further details below in association with FIG. 7.

A state (represented by a node) in the inter-window HMM 600 includes three different labels. In the particular embodiment shown in FIG. 6, the three labels are orderly presented as a first parent label, a second parent label, and a switch label that represents a switch of the order between the first parent label and the second parent label in the particular window, where the switching may be associated with phasing errors. While the order of presentation in the embodiment shown in FIG. 6 is the first parent label, the second parent label, and the switch label, other orders of presentation are also possible.

Each of the three labels in a state is represented by an integer value. For example, both the first parent label and the second parent label are selected from a set of K possible labels. A label is a classification of genetic data. For example, one possible way to classify genetic data is by ethnic origins of the individual, although other ways to classify genetic data are possible and are not necessarily based on or related to ethnic origins. If ethnic origins are used as classification, the set of K possible labels may be African, Asian, European, etc. or be German, Korean, Mexican, etc., depending on the granularity of the classification. A particular integral value represents one of the labels. For example, 1 may represent European while 2 may represent Asian.

The third label of a node, which is the switch label, may take a binary value (e.g., 1 or 0). The first binary value (e.g., 1) may represent that there is a switching of order of the first parent label and the second parent label while the second binary value (e.g., 0) may represent that there is no switching of order. A switch label represents a switching of order of the first parent label and the second parent label. In other words, a switch label represents that, for a particular state, the order of the first parent label and second parent label in the HMM is switched compared to the actual labels in the sample. Using the examples discussed in this paragraph as an illustration, the first node 602 of Window 1 in FIG. 6, which takes the values (1, 1, 0), may represent the state that Window 1 is labeled as European for both first parent label and second parent label and there is no switching of order between the two labels.

Likewise, the fourth node 604 of Window 1 in FIG. 6, which takes the values of (1, 2, 1), may represent the state that Window 1 is labeled as European for first parent label and Asian for second parent label but there is a switching of order between the two labels. In other words, due to one or more possible, but unobserved reasons such as a phasing error, the fourth node 604 in fact represents that Window 1 has Asian as first parent label and European for second parent label.

Using node 602 as an example to explain the concept of emission probability in the inter-window HMM 600, the emission probabilities here represent the likelihoods that Window 1 is observed in the sample genotype dataset to have a particular pair of phased haplotype datasets given the Window 1 should be labeled as having European origin for both first parent ethnicity and second parent ethnicity. Likewise, the transition probability from the node 602 to the node 606 represents the likelihood that a first segment of SNPs (corresponding to Window 1), which should be labeled as having European origin for both first and second parent ancestries, transitions to a second segment of SNPs (corresponding to Window 2) that should be labeled as having European origin for the first parent ethnicity and European origin for the second parent ethnicity, but there is a switching of first parent label and second parent label.

The plurality of nodes in each node group represents permutations of different possible first parent labels, second parent labels, and switch labels that can be assigned to a window. For each window, the inter-window HMM 600 may include a set of states corresponding to every ordered set of labels. Hence, the total number of states (T) can be K*K*2 (first parent labels K*second parent labels K*binary switch labels) for each window. For the particular embodiment shown in FIG. 6, there are three possible values of classification labels (i.e., K=3) and the switch label takes the value of either 1 or 0. Hence, there are 3*3*2=18 possible states (i.e., T=18). For simplicity, only some of the states are shown in FIG. 6 for each window. The states for a window w are denoted as U_{w}(p,q,z) where p is the value of the first parent label (e.g., p∈(1, 2, . . . , K)), q is the value of the second parent label (e.g., q∈(1, 2, . . . , K)), and z is the value of the switch label (e.g., z∈(0,1)). In this way, the set of labels (p,q,z) uniquely refers to each of the possible states T. Although FIG. 6 depicts K=3 labels, the number of labels K can be any natural integers.

The inter-window HMM 600 is a directional graph that represents a transition from a start state to an end state (not shown in FIG. 6) through a plurality of node groups that represent a plurality of windows. The start state 610 transitions to one of the T possible states of window 1 as illustrated by the arrows between the start state 610 and the respective T states of window 1. Each state in window 1 may transition to one of the possible states in window 2. A state U_{w}(p,q,z) in window w may transition to a state U_{w+1}(p′,q′,z′) in window w+1. The chromosome that corresponds to the window w is denoted as C(w) while the chromosome that corresponds to the window w+1 is denoted as C(w+1). If the window w and the window w+1 correspond to the same chromosome (i.e., C(w)=C(w+1)), then a state U_{w}(p,q,z) may be more likely to transition to a state U_{w+1}(p′,q′,z′) in window w+1 that corresponds to the same pair of labels (i.e., (p′,q′)=(p,q)) without switching than to a state in window w+1 that corresponds to a different pair of labels or to a state in window w+1 that corresponds to a switching of labels. This is because it is biologically unlikely that the sequences of SNPs in adjacent windows will correspond to different labels (e.g., correspond to different ancestral origin groups).

In some embodiments, the transition probability P(U_{w}(p,q,z), U_{w+1}(p′,q′,z′)) from a state U_{w}(p,q,z) to a state U_{w+1}(p′,q′,z′) is given by equation (1) below:

$$P ( {U}_{w} ( p , q , 𝓏 ) \to {U}_{w + 1} ( p ’ , q ’ , 𝓏 ’ ) ) = \{ \begin{matrix}\frac{{π}_{p ′}^{m} \times {π}_{q ′}^{f}}{2} if C ( w ) \neq C ( w + 1 ) \\ ( 1 - {τ}^{m} ) \times ( 1 - {τ}^{f} ) \times ( 1 - {τ}^{𝓏} ) if C ( w ) = C ( w + 1 ) , p = {p}^{′} , q = {q}^{′} , 𝓏 = {𝓏}^{′} \\ ( 1 - {τ}^{m} ) \times ( 1 - {τ}^{f} ) \times {τ}^{𝓏} if C ( w ) = C ( w + 1 ) , p = {p}^{′} , q = {q}^{′} , 𝓏 \neq {𝓏}^{′} \\ {τ}^{m} \times ( 1 - {τ}^{f} ) \times ( 1 - {τ}^{𝓏} ) \times \frac{{π}_{{p}^{′}}^{m}}{{\sum }_{1}^{{p}^{′} - 1} {π}_{k}^{m} + {\sum }_{{p}^{′} + 1}^{K} {π}_{k}^{m}} if C ( w ) = C ( w + 1 ) , p \neq {p}^{′} , q = {q}^{′} , 𝓏 = {𝓏}^{′} \\ {τ}^{m} \times ( 1 - {τ}^{f} ) \times {τ}^{𝓏} \times \frac{{π}_{{p}^{′}}^{m}}{{\sum }_{1}^{{p}^{′} - 1} {π}_{k}^{m} + {\sum }_{{p}^{′} + 1}^{K} {π}_{k}^{m}} if C ( w ) = C ( w + 1 ) , p \neq {p}^{′} , q = {q}^{′} , 𝓏 \neq {𝓏}^{′} \\ ( 1 - {τ}^{m} ) \times {τ}^{f} \times ( 1 - {τ}^{𝓏} ) \times \frac{{π}_{{q}^{′}}^{m}}{{\sum }_{1}^{{q}^{′} - 1} {π}_{k}^{f} + {\sum }_{{q}^{′} + 1}^{K} {π}_{k}^{f}} if C ( w ) = C ( w + 1 ) , p = {p}^{′} , q \neq {q}^{′} , 𝓏 = {𝓏}^{′} \\ ( 1 - {τ}^{m} ) \times {τ}^{f} \times {τ}^{𝓏} \times \frac{{π}_{{q}^{′}}^{m}}{{\sum }_{1}^{{q}^{′} - 1} {π}_{k}^{f} + {\sum }_{{q}^{′} + 1}^{K} {π}_{k}^{f}} if C ( w ) = C ( w + 1 ) , p = {p}^{′} , q \neq {q}^{′} , 𝓏 \neq {𝓏}^{′} \\ 0 if C ( w ) = C ( w + 1 ) , p \neq {p}^{′} , q \neq {q}^{′} .\end{matrix}$$

The symbol

$${π}_{k}^{m}$$

represents the label probability distribution of first parent label k over K different labels while

$${π}_{k}^{f}$$

represents the label probability distribution of second parent label k over K different labels. In some embodiments the label probability distributions may each correspond to a genome wide distribution, but in other embodiments the distributions may correspond to a portion of the genome. In some cases, the label probabilities over all different labels sum to unity

$$( i . e . , {\sum }_{k ∈ K} {π}_{k}^{m} = {\sum }_{k ∈ K} {π}_{k}^{f} = 1 ) .$$

The label probability distributions

$${π}_{k}^{m} and {π}_{k}^{f}$$

indicates the preference of parent 1 and parent 2, respectively, for K different labels. For example,

$${π}_{p ′}^{m}$$

is the probability of first parent label of window w+1 taking the value k=p′ over other possible values of labels K. C(w)=C(w+1) represents that the two windows correspond to the same chromosome. The label change probability τ^{m} represents the probability that first parent label will transition to a different label from window w to window w+1 (e.g., window w has a label of European while window w+1 has a label of Asian). In the embodiment that uses the equation above, the change of label depends on label probability

$${π}_{k}^{m} and {π}_{k}^{f} .$$

The label change probability τ^{ƒ} represents the probability that second parent label will transition to a different label from window w to window w+1. The label switch probability τ^{z} represents the probability that the order of first parent label and the second parent label is switched (i.e. the state will transition to the opposite z assignment between two windows.)

Hence, in the above equation, the first scenario represents that two windows are located in different chromosomes and the transition probability P(U_{w}(p,q,z), Uₓ₊₁(p′,q′,z′)) is equal to the first parent label probability of k=p′ times the second parent label probability of k=q′ divided by 2. The second scenario represents that the two windows are located in the same chromosome and there is no change in label or switch of label order. The transition probability in this scenario is equal to one minus the first parent label change probability τ^{m} (because the label either change or does not change) times one minus the second parent label change probability τ^{ƒ} times one minus label switch probability τ^{z}. Other scenarios are modeled similarly in the equation above.

The values of label probabilities

$$( {π}_{k}^{m} and {π}_{k}^{f} ) .$$

label change probabilities (τ^{m} and τ^{ƒ}), and the label switch probability (τ^{z}) are determined by the training of the inter-window HMM 600 based on a set of training data and, in some embodiments, additionally with the pair of haplotype datasets derived from an input sample genotype dataset X. The values of label probabilities

$$( {π}_{k}^{m} and {π}_{k}^{f} )$$

of different k may be represented in a vector form (also referred to as label probability vector). In some embodiments, the values of the label probability vector and the label change probabilities are calculated with a Baum-Welch algorithm. In some embodiments, it may be assumed that a transition from a state U_{w}(p,q,z) to another state U_{w+1}(p′,q′,z′) without any of the same labels p, q (i.e., both values of first parent label and second parent label change in a transition) are impossible. Hence, the transition probability for the last scenario in the equation above is zero in some embodiments. By omitting a transition for these low-probability transitions, the complexity of the inter-window HMM 600 may be reduced, thereby producing significant savings in time and computer processing requirements needed to determine labels.

If the window w+1 corresponds to a different chromosome than window w, then the state U_{w}(p,q,z) may transition to an inter-chromosome state 620, which, in turn, transitions to a state U_{w+1}(p′,q′,z′) in the next window w+1. Thus, if the window w+1 corresponds to a different chromosome than window w, the state U_{w}(p,q,z) may transition to a state U_{w+1}(p′,q′,z′) with a probability that is independent of the state U_{w}(p,q,z) at window w (i.e., independent of (p,q)) because of the intervening inter-chromosome state 620.

If window w is the final window (i.e., w=W), then the state U_{w}(p,q,z) in the window w transitions to an end state (not shown in FIG. 6). Each state U_{w}(p,q,z) in window w transitions to either a state Uₓ₊₁(p′,q′,z′) in window w+1, an inter-chromosome state 620, or an end state. FIG. 6 illustrates the possible outgoing transitions for each state U_{w}(p,q,z) with arrows. For example, in window 2 (and in all windows w in which the window w+1 is on the same chromosome), the state 606 U₂(1,2,1) may transition to the states U₃(1,1,0), U₃(1,1,1), U₃(1,2,0), U₃(1,2,1), etc. However, the state 606 U₂(1,2,1) may not transition to state U₃(3,3,0) because of both the first parent label and second parent label change in the transition. As such, no arrow connects the state 606 U₂(1,2,1) to the state U₃(3,3,0) in FIG. 6.

In FIG. 6, each node (representing a state of a window) is associated with an emission probability that represents a likelihood of the window is observed as having a particular pair of phased haplotype datasets given the window is in the hidden state represented by the node. The determination of the emission probability is based on genotype data of different reference panels and the input genotype dataset X through one or more intermediate steps that may include determinations of annotations, annotation products, and label pair probabilities. The details of the determination of the emission probability are discussed below.

Returning first to FIG. 2, the reference panel sample store 240 stores a set of reference panel samples of genotype datasets for each of the K labels. A reference panel for kth label is a collection of representative genetic datasets that belong to a community corresponding to kth label. For example, if the kth label represents a community of individual of an Asian reference panel, the reference panel samples in the kth-label reference panel are representative Asian genotype datasets. For more details on how reference panel samples may be identified and/or generated, U.S. Pat. No. 10,223,498, granted Mar. 5, 2019, entitled “Discovering Population Structure from Patterns of Identity-by-Descent,” is incorporated by reference herein for all purposes. The set of reference panel samples corresponding to the kth label (for k∈{1, . . . , K}) is referred to herein as Rₖ. Each reference panel sample R∈Rₖ in the store 240 may be phased diploid genotype dataset of L SNPs, R=(R₁, . . . , R_{L}), where each Rᵢ (for i∈{1, . . . , L}) is an SNP that is an ordered pair of binary alleles (i.e., (0,0), (0,1), (1,0) or (1,1)). At some sites of SNPs, there may be missing data. The labels may each correspond to a different origin population (e.g., an ethnic group), in which case each reference panel sample R may be a genotype data with a single origin from the kth origin population.

The possible labels may include both unadmixed labels and admixed labels. A collection of reference panel samples may be accessed. The collection may include a plurality of unadmixed genetic datasets and a plurality of admixed synthetic genetic datasets. An admixed synthetic genetic dataset may be associated with both an ethnic origin and a geographical origin. For an admixed population, the same ethnic origin but with different geographical origins may be regarded as a different label. For labeling an admixed individuals, at least some of the nodes in the inter-window HMM 600 may be labeled with a particular ethnic origin associated with an admixed population from a geographical origin. Other nodes in the inter-window HMM 600 may be labeled with another ethnic origin associated with the admixed population from the geographical origin. For example, in FIG. 6 shown, label 1 may be associated with Mexico-Native American while label 2 may be associated with Mexico-European.

When a population is overrepresented in the original data, there is a risk of this population disproportionately influencing the algorithm's outputs. To compensate, the weight assigned to the overrepresented population may be adjusted downward. The adjustment may reduce the impact on the final outcomes, providing room for lesser-represented populations to play a proportionate role in the results. If a population is underrepresented, its corresponding weight may be increased. This adjustment may provide that the underrepresented population has sufficient influence in determining the outcomes of the algorithm, thereby compensating for its lesser representation in the algorithm's original data. In some embodiments, the weight of the population may be adjusted from 1 to 0.99 or 1.01 depending on overrepresentation or underrepresentation.

Now referring to FIG. 7A, a flowchart depicting an example process 700 for calculating emission probabilities is illustrated, according to some embodiments. The process 700 may be performed by one or more engines of the computing server 130 illustrated in FIG. 2, such as the ethnicity estimation engine 245. The process 700 may be embodied as a software algorithm that may be stored as computer instructions that are executable by one or more processors. The instructions, when executed by the processors, cause the processors to perform various steps in the process 700. In various embodiments, the process 700 may include additional, fewer, or different steps. While various steps in process 700 may be performed with the use of computing server 130, each step may be performed by a different computing device.

In some embodiments, the computing server 130 may receive haplotype data of a training set (step 710). The haplotype data may be a sequence of alleles corresponding to individuals. Each sequence of haplotype data may include alleles corresponding to the L SNPs of the genotypes stored in the genetic data store 205, or some subset thereof. The reference panel sample store 240 stores a set of reference samples for each of the K labels. The set of reference panel samples corresponding to the kth label (for k∈{1, . . . , K}) is referred to herein as Rₖ. Each reference panel sample RER, in the store 240 may be an unphased diploid genotype of L SNPs, R=(R₁, . . . , R_{L}), where each Rᵢ (for i∈{1, . . . , L}) is an SNP that is either an unordered pair of binary alleles (i.e., (0,0), (0,1), or (1,1)) or missing data. The labels may each correspond to a different origin (e.g., an ethnic group), in which case each reference panel sample R may be a genotype from the kth origin population.

Some or all of the haplotype data may be phased haplotype data produced by the methods described in the PCT application entitled “Haplotype Phasing Modules” (international publication number WO 2016/061568 A1) which was filed on Oct. 19, 2015, and the U.S. application entitled “Clustering of matched segments to determine linkage of dataset in a database” (publication number 2021/0034647) which was filed on Jul. 23, 2020, which are both hereby incorporated by reference in their entirety. In alternate embodiments, some or all of the haplotype data may be phased haplotypes produced by PHASE, BEAGLE, HAPI-UR, SHAPEIT2, IMPUTE2, or some other phase estimation method. Based on the received haplotype data, the computing server 130 may build haploid Markov Models (MMs) for each window w (step 720). The haploid MMs may be stored in a haploid MM store. The computing server 130 may include the haploid MM store.

The computing server 130 may receive a set of reference panel samples Rₖ for each label k (for 1≤k≤K) (step 730). The set of reference panel samples Rₖ may be accessed from the reference panel sample store 240. Based on the set of reference panel samples Rₖ for label k and the haploid MMs for window w, computing server 130 may calculate a set of annotations A_{w}(k,u) of every label k and every state u in the window w (step 740). The annotations A_{w} may be stored in an annotation store. The computing server 130 may include the annotation store.

The computing server 130 may determine weight parameters P_{w} for the annotations (step 745). The weight parameters P_{w} may be determined and/or adjusted according to the process described in FIG. 3. The weight parameters P_{w} may be stored in the annotation store. The computing server 130 may access the weight parameters P_{w} from the annotation store. FIG. 7D shows examples of weight parameters associated with specific populations.

The computing server 130 may calculate annotation products L′_{w}(d, p) based on the annotations and the weight parameters (step 750). Based on the annotation products L′_{w}(d, p), the computing server 130 may calculate label probability distributions (step 760). Based on the label pair probability distributions E_{x,w}(p,q), the computing server 130 may calculate the emission probability for each node (step 770). For an admixed individual, at least some of the nodes in the inter-window HMM 600 may be assigned with probabilities that are calculated based on one or more synthetic genetic datasets.

### Annotation Determination

The discussion in this subsection corresponds to element 740 in FIG. 7A regarding calculation of annotation in association with the calculation of emission probabilities. The annotation A_{w}(k,u) is based on a calculation of the conditional probability of the haploid state u given the SNP sequence in the window w for the reference panel sample R that belongs to the set of reference panel samples Rₖ of the kth label. The calculation of the probability of the state u given reference panel sample R is based on the haploid MM for window w. For a given window w, label k, and state u, the annotation A_{w}(k,u) is equal to or positively correlated with the probability that a haplotype corresponding to label k includes the haploid state u in its path through window w. Equivalently, the annotation A_{w}(k,u) may be or may represent the expected proportion of haplotypes that include haploid state u in their corresponding paths for genotypes datasets selected from the set of reference panel samples Rₖ.

In one embodiment, annotations are determined using a forward-backward algorithm. For a reference panel sample R∈Rₖ, the forward-backward algorithm may be used to calculate a forward function ƒ_{R,w} and a backward function b_{R,w}. The forward function ƒ_{R,w}(u,v) may map the diploid state (u,v) at level d to the joint probability of the first d SNPs in window w of the reference panel sample R and the diploid state (u,v). That is, the output of the forward function ƒ_{R,w}(u,v) is the probability, based on the haploid MM for the window w, that a genotype dataset has the first d SNPs of R and that R corresponds to the state (u,v) at level d. Similarly, the backward function b_{R,w}(u,v) may map the diploid state (u,v) at level d to the joint probability of the last (D-d) SNPs in window w of the reference panel sample R and the state (u,v). The forward-backwards product, ƒ_{R,w}(u,v)×b_{R,w}(u,v), may be the joint probability of all the SNPs of the reference panel sample R in window w and the corresponding state (u,v). In some embodiments, the outputs of the forward function ƒ_{R,w} and the backward function b_{R,w} are proportional, but not necessarily equal to the probabilities of their respective diploid states.

The annotation A_{w}(k,u) for the label k and state u may be given by:

$${A}_{w} ( k , u ) = \frac{1}{\vert {R}_{k} \vert} {\sum }_{R ∈ {R}_{k}} \frac{1}{{b}_{R , w} ( {𝕊}_{w} , {𝕊}_{w} )} {\sum }_{v ∈ S t a tes In {Level}_{w} ( u )} {f}_{R , W} ( u , v ) \times {b}_{R , w} ( u , v ) \qquad ( 1 )$$

where |Rₖ| denotes the cardinality of the set Rₖ (i.e., the number of reference panel samples in Rₖ) and where StatesInLevel_{w}(u) refers to the set of haploid states in the same level as u (i.e., if u is in level d, then StatesInLevel_{w}(u) is the set of all states at level d). Because ( [symbol] _{w}, [symbol] _{w}) is the start state of the diploid HMM 300 for window w, b_{R,w}( [symbol] _{w}, [symbol] _{w}) is equal to the likelihood of the reference panel sample R.

By the definition of the conditional probability, ƒ_{R,w}(u,v)×b_{R,w}(u,v)/b_{R,w}( [symbol] _{w}, [symbol] _{w}) is the diploid state probability, i.e., the conditional probability that the path of a genotype dataset includes the state (u,v) in diploid HMM for window w given that the genotype dataset is a reference panel sample R. In some embodiments, the forward-backwards product ƒ_{R,w}(u,v)×b_{R,w}(u,v) and b_{R,w}( [symbol] _{w}, [symbol] _{w}) are calculated to be proportional, but not necessarily equivalent, to the likelihood of their respective diploid states. In such an embodiment, the diploid state probability ƒ_{R,w}(u,v)×b_{R,w}(u,v)/b_{R,w}( [symbol] _{w}, [symbol] _{w}) for reference panel sample R is still equivalent to the conditional probability that the path of the genotype includes the state (u,v) in the diploid HMM given the genotype R.

The summation of the diploid state ƒ_{R,w}(u,v)×b_{R,w}(u,v)/b_{R,w}( [symbol] _{w}, [symbol] _{w}) over all haploid states v in level d produces the marginal probability that the first haplotype (e.g., paternal, or maternal) is in haploid state u at level d given the reference panel sample R. The diploid state probabilities for a reference panel sample R may be summed over the set of diploid states that include the haploid state u (i.e., diploid states (u,v) and (v,u) for all haploid states v at the same level as the haploid state u) to produce a probability that the reference panel sample R corresponds to the haploid state u. Finally, the probabilities of u for each reference panel sample R may be combined to produce the annotation A_{w}(k,u). For example, A_{w}(k,u) may be the arithmetic average of the probabilities of the haploid state u for each reference panel sample R, therefore representing the expected proportion of reference panel samples in the set of reference panel samples Rₖ that include the state u in their respective paths. Stated differently, the annotation A_{w}(k,u) is the probability that the haploid state of a haplotype at a level d is haploid state u given that the haplotype corresponds to label k. In other alternatives, a different mathematical formulation other than arithmetic average may be used.

The annotations in the annotation store may be calculated prior to determining labels for potentially admixed genotype datasets. In some embodiments, the annotations are updated based on labels determined for phased potentially admixed genotype datasets that are input to the system through the process 700 described herein. In some embodiments, the annotations A_{w}(k,u) for a label k and window w may be iteratively improved by determining a probability that an admixed genotype dataset corresponds to a label k in window w and modifying the annotations A_{w}(k,u) accordingly.

### Annotation Product Determination

The discussion in this subsection may correspond to element 750 in FIG. 7A regarding calculation of annotation products in association with the calculation of emission probabilities.

Based on the annotations A_{w}(k,u) and the input sample genotype dataset X, which is divided into two phased haplotypes, x_{1,w} and x_{2,w}, each a sequence of alleles ∈{0,1} corresponding to the subsequence of SNPs in window w, the computing server 130 may calculate a label probability E_{x,w}(p) for each haplotype x∈{x_{1,w},x_{2,w}}, and each label p ∈{1, 2, . . . , K}, where K is the number of possible labels. If window w is a subsequence of D_{w} SNPs, the computing server 130 may determine a unique set of states {u_{x,w,0}, u_{s,w,1}, u₂, . . . , u_{x,w,Dw}} for a haplotype subsequence x in window w and the label probability for label p for a haplotype x is given by

$${E}_{x , w} ( p ) = \frac{1}{{D}_{w}} {\sum }_{d = 0}^{{D}_{w}} \frac{{A}_{w} ( p , {u}_{x , w , d} )}{{\sum }_{k = 1}^{K} {A}_{w} ( k , {u}_{x , w , d} )}$$

Based on the annotations A_{w}(k,u), the weight parameters P_{w} and the input sample genotype dataset X, the computing server 130 may calculate an expected annotation product L′_{x,w}(d,p) for level d and for label p (or label q). The expected annotation product L′_{x,w}(d,p) in each window w may be the probability that a stochastic phased haplotype of label p corresponds to the same phased state at level d as the input sample dataset given the haplotype sequence in window w. The expected annotation product L′_{x,w}(d,p) may be based on the annotations A_{w}(k,u) and the weight parameters P_{w}.

$${L}_{x , w}^{′} ( p , q ) = \frac{{f}_{x , w} ( u ) \times {b}_{x , w} ( u )}{{b}_{u , w} ( {𝕊}_{w} , {𝕊}_{w} )} \times {A}_{w} ( p , u ) \times {P}_{w}$$

FIGS. 7B and 7C show the evaluation results of the machine learning model. 0 means underrepresented and 2 means overrepresented. FIG. 7B shows the results of the machine learning model before the step of initiating origin-specific weight parameters. FIG. 7C shows the results of the machine learning model after the step of initiating origin-specific weight parameters. For example, in FIG. 7C, all origins mostly fall near the mark 1, which may be expected.

### Computing Machine Architecture

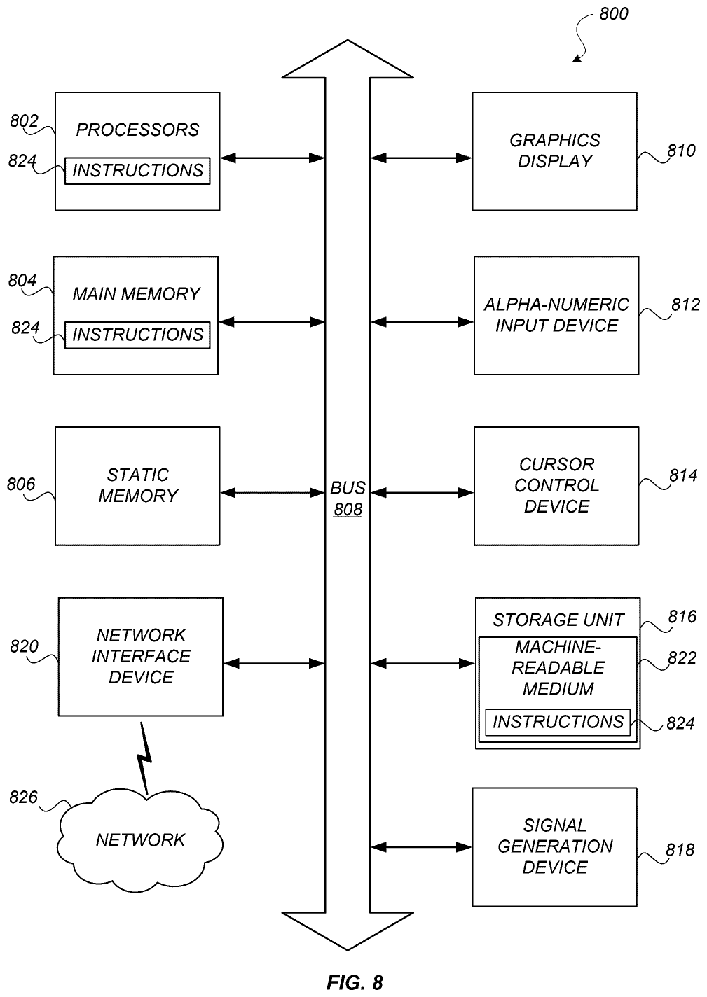

FIG. 8 is a block diagram illustrating components of an example computing machine that is capable of reading instructions from a computer-readable medium and execute them in a processor (or controller). A computer described herein may include a single computing machine shown in FIG. 8, a virtual machine, a distributed computing system that includes multiple nodes of computing machines shown in FIG. 8, or any other suitable arrangement of computing devices.

By way of example, FIG. 8 shows a diagrammatic representation of a computing machine in the example form of a computer system 800 within which instructions 824 (e.g., software, source code, program code, expanded code, object code, assembly code, or machine code), which may be stored in a computer-readable medium for causing the machine to perform any one or more of the processes discussed herein and may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 8 may correspond to any software, hardware, or combined components shown in FIGS. 1 and 2, including but not limited to, the client device 110, the computing server 130, and various engines, interfaces, terminals, and machines shown in FIG. 2. While FIG. 8 shows various hardware and software elements, each of the components described in FIGS. 1 and 2 may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 824 that specify actions to be taken by that machine. Further, while only a single machine is illustrated, the term “machine” and “computer” may also be taken to include any collection of machines that individually or jointly execute instructions 824 to perform any one or more of the methodologies discussed herein.

The example computer system 800 includes one or more processors 802 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 800 may also include a memory 804 that store computer code including instructions 824 that may cause the processors 802 to perform certain actions when the instructions are executed, directly or indirectly by the processors 802. Instructions can be any directions, commands, or orders that may be stored in different forms, such as equipment-readable instructions, programming instructions including source code, and other communication signals and orders. Instructions may be used in a general sense and are not limited to machine-readable codes. One or more steps in various processes described may be performed by passing through instructions to one or more multiply-accumulate (MAC) units of the processors.

One or more methods described herein improve the operation speed of the processors 802 and reduces the space required for the memory 804. For example, the database processing techniques and machine learning methods described herein reduce the complexity of the computation of the processors 802 by applying one or more novel techniques that simplify the steps in training, reaching convergence, and generating results of the processors 802. The algorithms described herein also reduces the size of the models and datasets to reduce the storage space requirement for memory 804.

The performance of certain operations may be distributed among more than one processor, not only residing within a single machine, but deployed across a number of machines. In some example embodiments, the one or more processors or processor-implemented modules may be located in a single geographic location (e.g., within a home environment, an office environment, or a server farm). In other example embodiments, one or more processors or processor-implemented modules may be distributed across a number of geographic locations. Even though the specification or the claims may refer to some processes being performed by a processor, this should be construed to include a joint operation of multiple distributed processors.

The computer system 800 may include a main memory 804, and a static memory 806, which are configured to communicate with each other via a bus 808. The computer system 800 may further include a graphics display unit 810 (e.g., a plasma display panel (PDP), a liquid crystal display (LCD), a projector, or a cathode ray tube (CRT)). The graphics display unit 810, controlled by the processors 802, displays a graphical user interface (GUI) to display one or more results and data generated by the processes described herein. The computer system 800 may also include alphanumeric input device 812 (e.g., a keyboard), a cursor control device 814 (e.g., a mouse, a trackball, a joystick, a motion sensor, or other pointing instruments), a storage unit 816 (a hard drive, a solid-state drive, a hybrid drive, a memory disk, etc.), a signal generation device 818 (e.g., a speaker), and a network interface device 820, which also are configured to communicate via the bus 808.

The storage unit 816 includes a computer-readable medium 822 on which is stored instructions 824 embodying any one or more of the methodologies or functions described herein. The instructions 824 may also reside, completely or at least partially, within the main memory 804 or within the processor 802 (e.g., within a processor's cache memory) during execution thereof by the computer system 800, the main memory 804 and the processor 802 also constituting computer-readable media. The instructions 824 may be transmitted or received over a network 826 via the network interface device 820.

While computer-readable medium 822 is shown in an example embodiment to be a single medium, the term “computer-readable medium” should be taken to include a single medium or multiple media (e.g., a centralized or distributed database, or associated caches and servers) able to store instructions (e.g., instructions 824). The computer-readable medium may include any medium that is capable of storing instructions (e.g., instructions 824) for execution by the processors (e.g., processors 802) and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

### ADDITIONAL CONSIDERATIONS

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Any feature mentioned in one claim category, e.g. method, can be claimed in another claim category, e.g. computer program product, system, storage medium. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter may include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein may be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines may be embodied in software, firmware, hardware, or any combinations thereof.

Any of the steps, operations, or processes described herein may be performed or implemented with one or more hardware or software engines, alone or in combination with other devices. In some embodiments, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure may describe a process that includes multiple steps sequentially with arrows present in a flowchart, the steps in the process do not need to be performed in the specific order claimed or described in the disclosure. Some steps may be performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, the use of a singular form of a noun may imply at least one element even though a plural form is not used.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

The following applications are incorporated by reference in their entirety for all purposes: (1) U.S. Pat. No. 10,679,729, entitled “Haplotype Phasing Models,” granted on Jun. 9, 2020, (2) U.S. Pat. No. 10,223,498, entitled “Discovering Population Structure from Patterns of Identity-By-Descent,” granted on Mar. 5, 2019, (3) U.S. Pat. No. 10,720,229, entitled “Reducing Error in Predicted Genetic Relationships,” granted on Jul. 21, 2020, (4) U.S. Pat. No. 10,558,930, entitled “Local Genetic Ethnicity Determination System,” granted on Feb. 11, 2020, (5) U.S. Pat. No. 10,114,922, entitled “Identifying Ancestral Relationships Using a Continuous Stream of Input,” granted on Oct. 30, 2018, (6) U.S. Pat. No. 11,429,615, entitled “Linking Individual Datasets to a Database,” granted on Aug. 30, 2022, (7) U.S. Pat. No. 10,692,587, entitled “Global Ancestry Determination System,” granted on Jun. 23, 2020, and (8) U.S. Patent Application Publication No. US 2021/0034647, entitled “Clustering of Matched Segments to Determine Linkage of Dataset in a Database,” published on Feb. 4, 2021.

1. A computer-implemented method for improving training of a machine learning model, the computer-implemented method comprising:
generating a plurality of reference panels for a plurality of data-inheritance origins, each reference panel corresponding to a data-inheritance origin corresponding to a population and comprising reference-panel datasets genetic markers representative of the data-inheritance origin;
constructing a plurality of simulated inheritance trees, wherein each simulated inheritance tree corresponds to a population and is built using the reference-panel datasets that are selected from the reference panel corresponding to the population, wherein building a simulated inheritance tree comprises simulating meiosis and recombination events based on the genetic markers of the reference-panel datasets to generate a plurality of simulated inheritance datasets of simulated descendant named entities;
generating plurality of simulated inheritance datasets representing a plurality of simulated named entities, each representing a simulated descendant named entity in one of the simulated inheritance trees; and
training a machine learning model that is configured to determine inheritance labels of an inheritance dataset, wherein training the machine learning model comprises:
initiating origin-specific weight parameters in the machine learning model;
applying the plurality of simulated inheritance datasets as training samples;
applying the machine learning model to predict inheritance labels of the training samples;
comparing predicted inheritance labels to actual labels obtained from the plurality of simulated inheritance trees; and
adjusting the origin-specific weight parameters based on label comparisons.

2. The computer-implemented method of claim 1, wherein generating the plurality of reference panels for the plurality of data-inheritance origins comprises:
filtering a plurality of candidates based on inheritance labels;
identifying, for each candidate, a number of the named entities in a particular data-inheritance origin whose inheritance datasets match the inheritance dataset of the candidate; and
selecting a candidate to be added to the reference-panel datasets based on the number of matched named entities that correspond to the candidate compared to numbers of matched named entities of other candidates.

3. The computer-implemented method of claim 1, wherein generating the plurality of reference panels for the plurality of data-inheritance origins comprises:
generating a candidate pool that include a plurality of candidates based on inheritance labels related to the particular origin;
determining that one of the candidates has a first number of matches associated with a particular origin and a second number of matches associated with a second origin, the second number of matches exceeding a threshold; and
removing said one of the candidates from the candidate pool.

4. The computer-implemented method of claim 1, wherein a particular simulated inheritance tree is associated with a geographical location, wherein constructing the particular simulated inheritance tree comprises:
accessing a population composition of the geographical location, the population composition comprising information related to percentage of named entities with the plurality of origins;
sampling, based on the population composition of the geographical location, reference-panel datasets from the plurality of reference panels for the plurality of origins; and
representing sampled reference-panel datasets in nodes of the particular simulated inheritance tree.

5. The computer-implemented method of claim 4, wherein the population composition comprising generation-specific composition, and wherein representing the sampled reference-panel datasets in the nodes of the particular simulated inheritance tree comprises:
selecting placements of the sampled reference-panel datasets based on the generation-specific composition.

6. The computer-implemented method of claim 1, wherein generating a particular simulated inheritance dataset representing a particular simulated named entity in a particular simulated inheritance tree comprises:
treating the particular simulated named entity as a descendant named entity of the reference-panel datasets that are placed in the particular simulated inheritance tree;
simulating a plurality of inheritance events; and
generating the particular simulated inheritance dataset of the particular simulated named entity based on the plurality of inheritance events.

7. The computer-implemented method of claim 1, wherein the machine learning model is a hidden Markov model with windows that represent segments of inheritance data, each window comprising a plurality of nodes and each node representing an origin, wherein the origin-specific weight parameters are associated with weights of the plurality of nodes.

8. The computer-implemented method of claim 1, wherein adjusting the origin-specific weight parameters comprises:
comparing the predicted inheritance labels to the actual labels to identifying under-represented origins and over-represented origins;
for an under-represented origin, increasing a value of the origin-specific weight parameter corresponding to the under-represented origin; and
for an over-represented origin, decreasing a value of the origin-specific weight parameter corresponding to the over-represented origin.

9. The computer-implemented method of claim 1, wherein the actual labels obtained from the plurality of simulated inheritance trees are generated by:
dividing a particular simulated inheritance dataset in a particular training sample into a plurality of windows;
examining how a segment of the particular simulated inheritance dataset in a particular window is inherited in a particular simulated inheritance tree;
identifying a reference-panel named entity in the particular inheritance tree who passes down the segment to the simulated inheritance dataset;
determining an origin label of said reference-panel named entity; and
using the origin label as the actual label.

10. The computer-implemented method of claim 1, wherein the training samples comprises admixed named entities that are simulated from plurality of simulated inheritance trees and non-admixed named entities that are sampled from actual user datasets.

11. A system comprising:
one or more processors; and
memory configured to store instructions, wherein the instructions, when executed by the one or more processors, cause the one or more processors to perform steps comprising:
generating a plurality of reference panels for a plurality of data-inheritance origins, each reference panel corresponding to a data-inheritance origin corresponding to a population and comprising reference-panel datasets genetic markers representative of the data-inheritance origin;
constructing a plurality of simulated inheritance trees, wherein each simulated inheritance tree corresponds to a population and is built using the reference-panel datasets that are selected from the reference panel corresponding to the population, wherein building a simulated inheritance tree comprises simulating meiosis and recombination events based on the genetic markers of the reference-panel datasets to generate a plurality of simulated inheritance datasets of simulated descendant named entities;
generating plurality of simulated inheritance datasets representing a plurality of simulated named entities, each representing a simulated descendant named entity in one of the simulated inheritance trees; and
training a machine learning model that is configured to determine inheritance labels of an inheritance dataset, wherein training the machine learning model comprises:
initiating origin-specific weight parameters in the machine learning model;
applying the plurality of simulated inheritance datasets as training samples;
applying the machine learning model to predict inheritance labels of the training samples;
comparing predicted inheritance labels to actual labels obtained from the plurality of simulated inheritance trees; and
adjusting the origin-specific weight parameters based on label comparisons.

12. The system of claim 11, wherein generating the plurality of reference panels for the plurality of data-inheritance origins comprises:
filtering a plurality of candidates based on inheritance labels;
identifying, for each candidate, a number of the named entities in a particular data-inheritance origin whose inheritance datasets match the inheritance dataset of the candidate; and
selecting a candidate to be added to the reference-panel datasets based on the number of matched named entities that correspond to the candidate compared to numbers of matched named entities of other candidates.

13. The system of claim 11, wherein generating the plurality of reference panels for the plurality of data-inheritance origins comprises:
generating a candidate pool that include a plurality of candidates based on inheritance labels related to the particular origin;
determining that one of the candidates has a first number of matches associated with a particular origin and a second number of matches associated with a second origin, the second number of matches exceeding a threshold; and
removing said one of the candidates from the candidate pool.

14. The system of claim 11, wherein a particular simulated inheritance tree is associated with a geographical location, wherein constructing the particular simulated inheritance tree comprises:
accessing a population composition of the geographical location, the population composition comprising information related to percentage of named entities with the plurality of origins;
sampling, based on the population composition of the geographical location, reference-panel datasets from the plurality of reference panels for the plurality of origins; and
representing sampled reference-panel datasets in nodes of the particular simulated inheritance tree.

15. The system of claim 14, wherein the population composition comprising generation-specific composition, and wherein representing the sampled reference-panel datasets in the nodes of the particular simulated inheritance tree comprises:
selecting placements of the sampled reference-panel datasets based on the generation-specific composition.

16. The system of claim 11, wherein generating a particular simulated inheritance dataset representing a particular simulated named entity in a particular simulated inheritance tree comprises:
treating the particular simulated named entity as a descendant named entity of the reference-panel datasets that are placed in the particular simulated inheritance tree;
simulating a plurality of inheritance events; and
generating the particular simulated inheritance dataset of the particular simulated named entity based on the plurality of inheritance events.

17. The system of claim 11, wherein the machine learning model is a hidden Markov model with windows that represent segments of inheritance data, each window comprising a plurality of nodes and each node representing an origin, wherein the origin-specific weight parameters are associated with weights of the plurality of nodes.

18. The system of claim 11, wherein adjusting the origin-specific weight parameters comprises:
comparing the predicted inheritance labels to the actual labels to identifying under-represented origins and over-represented origins;
for an under-represented origin, increasing a value of the origin-specific weight parameter corresponding to the under-represented origin; and
for an over-represented origin, decreasing a value of the origin-specific weight parameter corresponding to the over-represented origin.

19. The system of claim 11, wherein the actual labels obtained from the plurality of simulated inheritance trees are generated by:
dividing a particular simulated inheritance dataset in a particular training sample into a plurality of windows;
examining how a segment of the particular simulated inheritance dataset in a particular window is inherited in a particular simulated inheritance tree;
identifying a reference-panel named entity in the particular inheritance tree who passes down the segment to the simulated inheritance dataset;
determining an origin label of said reference-panel named entity; and
using the origin label as the actual label.

20. A non-transitory computer readable medium for storing computer code comprising instructions, when executed by one or more computer processors, causing one or more computer processors to perform steps comprising:
generating a plurality of reference panels for a plurality of data-inheritance origins, each reference panel corresponding to a data-inheritance origin corresponding to a population and comprising reference-panel datasets genetic markers representative of the data-inheritance origin;
constructing a plurality of simulated inheritance trees, wherein each simulated inheritance tree corresponds to a population and is built using the reference-panel datasets that are selected from the reference panel corresponding to the population, wherein building a simulated inheritance tree comprises simulating meiosis and recombination events based on the genetic markers of the reference-panel datasets to generate a plurality of simulated inheritance datasets of simulated descendant named entities;
generating plurality of simulated inheritance datasets representing a plurality of simulated named entities, each representing a simulated descendant named entity in one of the simulated inheritance trees; and
training a machine learning model that is configured to determine inheritance labels of an inheritance dataset, wherein training the machine learning model comprises:
initiating origin-specific weight parameters in the machine learning model;
applying the plurality of simulated inheritance datasets as training samples;
applying the machine learning model to predict inheritance labels of the training samples;
comparing predicted inheritance labels to actual labels obtained from the plurality of simulated inheritance trees; and
adjusting the origin-specific weight parameters based on label comparisons.
